(19) United States
(12) Patent Application Publication
(10) Pub. No.: US 2007/0259894 A1
(43) Pub. Date: Nov. 8, 2007

(54) Use of Atazanavir for Improving the Pharmacokinetics of Drugs Metabolized by Ugt1a1

(76) Inventor: Kelem Kassahun, Collegeville, PA (US)

Correspondence Address: MERCK AND CO., INC, P O BOX 2000, RAHWAY, NJ, 07065-0907, US

(21) Appl. No.: 11/792,189

(22) PCT Filed: Dec. 2, 2005
(86) PCT No.: PCT/US05/43782

§ 371 Date: May 31, 2007

Related U.S. Application Data
(60) Provisional application No. 60/632,945, filed on Dec. 3, 2004.

(51) Int. Cl. A61K 31/519; A61K 31/506; A61P 31/00; A61P 43/00; C07D 239/20; C07D 239/70

(52) U.S. Cl. 514/259.4; 514/269; 544/282; 544/319

(57) ABSTRACT

A method for improving the pharmacokinetics of an orally administered drug that is directly metabolized by UGT1A1 comprises orally administering to a mammal in need of treatment with the drug a combination of the drug or a pharmaceutically acceptable salt thereof and atazanavir or a pharmaceutically acceptable salt thereof.

This application claims the benefit of U.S. Provisional Application No. 60/632,945 (filed Dec. 3, 2004), the disclosure of which is hereby incorporated by reference in its entirety.

### FIELD OF THE INVENTION

The present invention is directed to a method for improving the pharmacokinetics of orally administered drugs that are metabolized by LTDP-glucuronosyl-transferase isoform 1A1 (UGT1A1), wherein the drugs are administered in combination with atazanavir. The present invention is also directed to methods for the inhibition of HIV integrase, for the treatment and prophylaxis of HIV infection, and for the treatment, prophylaxis, and delay in the onset of A-IDS, wherein the methods involve oral administration of an HIV integrase inhibitor metabolized by UGT1A1 in combination with atazanavir.

### BACKGROUND OF THE INVENTION

The UDP-glucuronosyltransferases (UGTs) are a family of enzymes that catalyze the glucuronidation of endogenous and xenobiotic chemicals; i.e., UGTs catalyze the transfer of a glucuronic acid group from the cofactor uridine diphosphate-glucuronic acid to a substrate. The transfer is generally to a nucleophilic O, N or S heteroatom. Substrates include xenobiotics which have been functionalized by Phase I reactions (e.g., P450 dependent oxidative metabolism), as well as endogenous compounds such as bilirubin, steroid hormones, and thyroid hormones. Although glucuronidation is generally classified as Phase II metabolism—the phase occurring after P450 dependent oxidative metabolism—many compounds do not require prior oxidation because they already possess functional groups that can be glucuronidated. Products of glucuronidation are excreted in urine if the molecular weight of the substrate is low (less than about 250 grams), whereas larger glucuronidated substrates are excreted in bile.

The UGTs play a key role in several important metabolic functions such as: elimination of drugs (e.g., non-steroidal anti-inflammatories, opioids, antihistamines, antipsychotics and antidepressants); detoxification of environmental contaminants such as benzo(a)pyrenes; regulation of hormone levels for androgens, estrogens, progestins, and retinoids; and elimination of the heme degradation product bilirubin.

UGTs are located in the microsomes of liver, kidney, intestine, skin, brain, spleen, and nasal mucosa, where they are on the same side of the endoplasmic reticulum membrane as cytochrome P450 enzymes and flavin-containing monooxygenases, and therefore are ideally located to access products of Phase I drug metabolism. UGTs involved in drug metabolism are encoded by two gene families, UGT1 and UGT2. The members of the UGT1 family that are expressed in human liver, where the majority of xenobiotic metabolism takes place, include UGT1A1, 1A3, 1A4, 1A6, and 1A9. UDP-glucuronosyl-transferase isoform 1A1 (UGT1A1) catalyzes the glucuronidation of bilirubin.

Some orally administered drugs, including certain HIV integrase inhibitors, are directly metabolized by UGT1A1, which can result in unfavorable pharmacokinetics and the need for more frequent and/or higher doses than would otherwise be necessary or desirable. The need for frequent dosing (e.g., 3 or more doses per day) can result in intentional or inadvertent patient non-compliance with the drug regimen. The use of higher doses can result in an increase in adverse reactions and/or toxic effects. Administration of such drugs with an agent that inhibits UGT1A1 metabolism can improve the pharmacokinetics of the drug which can permit a reduction in the dosing frequency. Improved pharmacokinetics resulting from co-administration with a UGT1A1 inhibitor can also permit the use of a lower dose which can reduce or eliminate the occurrence and/or severity of adverse reactions and toxic effects. Accordingly, there is a need for the discovery of compounds which can improve the pharmacokinetics of drugs metabolized by UGT1A1.

The following references are of interest as background:

US 2003/0215462 A1 discloses methods for increasing the bioavailability of certain orally administered pharmaceutical compounds by co-administering the compounds with UDP-glucuronosyltransferase inhibitors.

WO 03/35076 and the corresponding US 2005/0075356 each disclose certain 5,6-dihydroxypyrimidine-4-carboxamides as HIV integrase inhibitors, and WO 03/35077 and the corresponding US2005/0025774 each disclose certain N-substituted 5-hydroxy-6-oxo-1,6-dihydroxypyrimidine-4-carboxamides as HIV integrase inhibitors. Each of these references also discloses the use of the carboxamide compounds described therein in combination with one or more agents useful in the treatment of HIV infection or AIDS, wherein atazanavir is included in a list of suitable agents.

WO 2004/058756 discloses certain hydroxy-tetrahydropyridopyrimidinone carboxamides and related carboxamides as HIV integrase inhibitors. The reference also discloses the use of the carboxamide compounds described therein in combination with one or more agents useful in the treatment of HI infection or AIDS, and notes that suitable agents includes those listed in a table in WO 02/30930 which table includes atazanavir.

WO 2005/087768 discloses certain hydroxy polyhydro-2,6-naphthyridine dione compounds as HIV integrase inhibitors. The reference also discloses the use of the compounds in combination with one or more agents useful in the treatment of HIV infection or AIDS, and notes that atazanavir is among the suitable agents.

### SUMMARY OF THE INVENTION

It has been discovered that co-administration of atazanavir with a drug that is directly metabolized by UGT1A1 can provide an improvement in the pharmacokinetics of the drug. More particularly, the present invention includes a method for improving the pharmacokinetics of an orally administered drug that is directly metabolized by UGT1A1 which comprises orally administering to a mammal (especially a human) in need of treatment with the drug an effective amount of a combination of the drug or a pharmaceutically acceptable salt thereof and atazanavir or a pharmaceutically acceptable salt thereof.

Various embodiments, aspects and features of the present invention are either further described in or will be apparent from the ensuing description, examples and appended claims.

### BRIEF DESCRIPTION OF THE DRAWINGS

FIG. 1 is the X-ray powder diffraction pattern for the potassium salt of Compound A as prepared in Example 2.

FIG. 2 is the DSC curve for the potassium salt of Compound A as prepared in Example 2.

### DETAILED DESCRIPTION OF THE INVENTION

The present invention involves orally administering an effective amount of a combination of a drug that is directly metabolized by UGT1A1 and atazanavir. It is understood that the drug and atazanavir can be administered separately or together. When administered separately, they can be given concurrently or at different times (e.g., alternately). When administered together, they can be administered as separate compositions which can be packaged together or separately, or they can be administered as a single composition.

Drugs suitable for use in the present invention are those compounds for which UGT1A1-mediated metabolism is significant. In this context, “significant” means that at least about 20% of the orally administered drug is directly metabolized by UGT1A1. Drugs particularly suitable for use in the method of the present invention are those for which the primary route of metabolism following oral administration is direct metabolism by UGT1A1. The term “direct metabolism” and variants thereof (e.g., “directly metabolized”) mean herein that the metabolism involves direct glucuronidation of the drug; i.e., there is essentially no prior Phase I-type oxidation of the drug.

Atazanavir (also identified as BMS-232632) is an azapeptide inhibitor of HIV-1 protease effective for treating HIV infection. Atazanavir has the structural formula: 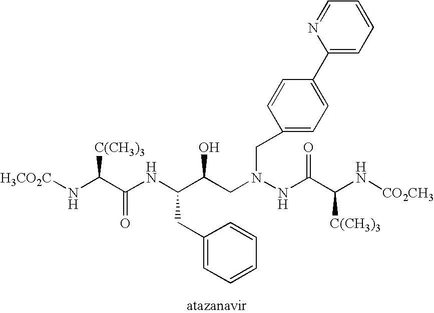
and its chemical name is [3S-(3R*, 8′R*, 9′R*, 12R*)]-3,12-bis(1,1-dimethylethyl)-8-hydroxy-4,11-dioxo-9-(phenylmethyl)-6-[[4-(2-pyridinyl)phenylmethyl]2,5,6,10,13-pentaazatetradecanedioic] acid, dimethyl ester. Atazanavir sulfate is approved for use in treating HIV infection and is available in capsule form under the tradename REYATAZ™ (Bristol-Myers Squibb). Atazanavir is disclosed in U.S. Pat. No. 5,849,911 and atazanavir sulfate is disclosed in U.S. Pat. No. 6,087,383. The 2004 edition of the Physician's Desk Reference (see p. 1082) discloses that atazanavir is an inhibitor of UDP-glucuronosyltransferase isoform 1A1 (UGT1A1).

An improvement in the pharmacokinetics (PK) of a drug means herein an increase in one or more of the following PK parameters as a result of co-administration of the drug with atazanavir compared to the corresponding value obtained by administration of the drug in the absence of atazanavir: peak plasma concentration (Cₘₐₓ), the trough plasma concentration (Cₘᵢₙ), the amount of drug in the bloodstream as measured by the area under the curve of plasma concentration versus time (AUC₀₋ₗₐₛₜ, where “last” refers to the time of last sampling—e.g., 24 hours), and half-life (T_{1/2}).

The drug and atazanavir can each independently and alternatively be administered in the form of a pharmaceutically acceptable salt. The term “pharmaceutically acceptable salt” refers to a salt which possesses the effectiveness of the parent agent and which is not biologically or otherwise undesirable (e.g., is neither toxic nor otherwise deleterious to the recipient thereof). Suitable salts include acid addition salts which may, for example, be formed by mixing a solution of the parent agent with a solution of a pharmaceutically acceptable acid such as hydrochloric acid, sulfuric acid, acetic acid, trifluoroacetic acid, or benzoic acid. If the drug carries an acidic moiety (e.g., —COOH or a phenolic group), pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. A preferred salt form of atazanavir is atazanavir sulfate, which is disclosed in U.S. Pat. No. 6,087,383.

Unless stated otherwise, references herein to amounts of drugs and/or amounts of atazanavir are to the amounts of their free, non-salt forms.

The term “effective amount” in reference to a combination employed in the present invention refers to the co-administration of the UGT1A1-metabolized drug and atazanavir in amounts suitable to elicit the biological or medicinal response to the drug that is being sought by the researcher, medical doctor, or other clinician. The effective amount refers to a “therapeutically effective amount”; i.e., co-administration of the UGT1A1-metabolized drug and atazanavir in amounts that result in the alleviation of the symptoms of the disease or condition being treated by the drug. The effective amount also refers to a “prophylactically effective amount”; i.e., co-administration of the drug and atazanavir in amounts that result in prophylaxis of the symptoms of the disease or condition being prevented by the drug. The term also includes herein the amount of active compound sufficient to inhibit an enzyme (e.g., HIV integrase) and thereby elicit the response being sought (i.e., an “inhibition effective amount”).

The drug and atazanavir can be co-administered in any proportion in the present invention, provided that the desired biological or medicinal response to the drug is achieved. For example, the drug can be co-administered in an amount which, if the amount were administered alone, would not achieve the desired response (e.g., unsatisfactory PK values for the drug and/or an unsatisfactory drug circulation level resulting in little or no efficacy) but which, as a result of co-administration with atazanavir, can achieve the desired response. As another example, the drug can be co-administered in an amount which, if it were administered alone, would achieve a suitable response (e.g., PK values and/or circulation level that achieve efficacy) but which, as a result of co-administration with atazanavir, is more effective (i.e., higher PK values such as higher AUC₀₋ₗₐₛₜ and/or higher Cₘᵢₙ, or higher circulation level).

A first embodiment of the present invention is the method for improving the PK of an orally administered drug directly metabolized by UGT1A1 as originally set forth above (i.e., as set forth in the Summary of the Invention), wherein atazanavir is administered in the combination in an amount sufficient to improve the pharmacokinetics of the drug by at least about 10% with respect to the pharmacokinetics of the drug administered in the absence of atazanavir (e.g., a 10% improvement in AUC₀₋ₗₐₛₜ or Cₘᵢₙ or Cₘₐₓ or T_{1/2}, or a combination thereof).

A second embodiment of the present invention is the method for improving PK as originally set forth above or as set forth in the preceding embodiment, wherein the mammal in need of treatment with the drug is a human.

A third embodiment of the present invention is the method for improving PK as originally set forth above or as set forth the first embodiment, wherein the mammal in need of treatment with the drug is a human, and the drug that is directly metabolized by UGT1A1 is selected from the group consisting of ezetimibe, raloxifene, estradiol, and pharmaceutically acceptable salts thereof. Ezetimibe selectively inhibits the intestinal absorption of cholesterol and is the active ingredient in ZETIA™ tablets (available from Merck-Schering Plough Pharmaceuticals). Ezetimibe and simvastatin are the active ingredients in VYTORIN™ tablets (available from Merck-Schering Plough Pharmaceuticals). Ezetimibe is disclosed in U.S. Pat. No. 5,846,966 and US Reissue 37721. Raloxifene is a selective estrogen receptor modulator. Raloxifene hydrochloride is the active ingredient in EVISTA® tablets (available from Eli Lilly) which is indicated for the treatment and prevention of osteoporosis in postmenopausal women. Raloxifene is disclosed in U.S. Pat. No. 6,458,811. Estradiol is the active ingredient in several products approved for treating various diseases and conditions such as vuval and vaginal atrophy, osteoporosis, and advanced prostate cancer.

A fourth embodiment of the present invention is the method for improving PK as originally set forth above or as set forth in either the first or second embodiment, wherein the drug that is directly metabolized by UGT1A1 is an HIV integrase inhibitor.

A fifth embodiment of the present invention is the method for improving PK as originally set forth above or as set forth in either the first or second embodiment wherein the drug that is directly metabolized by UGT1A1 is a compound of Formula I, or a pharmaceutically acceptable salt thereof: 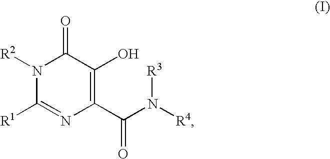
wherein R¹ is C₁₋₆ alkyl substituted with:
(1) N(R^{A})—C(═O)—N(R^{C})R^{D},
(2) N(R^{A})—C(═O)—C₁₋₆ alkylene-N(R^{C})R^{D},
(3) N(R^{A})SO₂R^{B},
(4) N(R^{A})SO₂N(R^{C})R^{D},
(5) N(R^{A})—C(═O)—C₁₋₆ alkylene-SO₂R^{B},
(6) N(R^{A})—C(═O)—C₁₋₆ alkylene-SO₂N(R^{C})R^{D},
(7) N(R^{A})C(═O)C(═O)N(R^{C})R^{D},
(8) N(R^{A})—C(═O)-HetA,
(9) N(R^{A})C(═O)C(═O)-HetA, or
(10) HetB;

R² is —C₁₋₆ alkyl;

or alternatively R¹ and R² are linked together such that the compound of Formula I is a compound of Formula II:

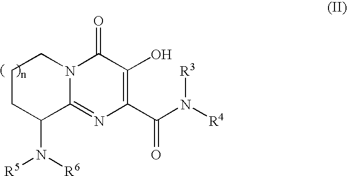

R³ is —H or —C₁₋₆ alkyl;

R⁴ is C₁₋₆ alkyl substituted with an aryl (e.g., phenyl), which is optionally substituted with from 1 to 4 substituents each of which is independently halogen, —OH, —C₁₋₄ alkyl, —C₁₋₄ alkyl-OR^{A}, —C₁₋₄ haloalkyl, —O—C₁₋₄ alkyl, —O—C₁₋₄ haloalkyl, —CN, —NO₂, —N(R^{A})R^{B}, —C₁₋₄ alkyl-N(R^{A})R^{B}, —C(═O)N(R^{A})R^{B}, —C(═O)R^{A}, —CO₂R^{A}, —C₁₋₄ alkyl-CO₂R^{A}, —OCO₂R^{A}, —SR^{A}, —S(═O)R^{A}, —SO₂R^{A}, —N(R^{A})SO₂R^{B}, —SO₂N(R^{A})R^{B}, —N(R^{A})C(═O)R^{B}, —N(R^{A})CO₂R^{B}, —C₁₋₄ alkyl-N(R^{A})CO₂R^{B}, methylenedioxy attached to two adjacent ring carbon atoms, phenyl, or —C₁₋₄ alkyl-phenyl;

R⁵ is:
(1) N(R^{A})—C(═O)—N(R^{C})R^{D},
(2) N(R^{A})—C(═O)—C₁₋₆ alkylene-N(R^{C})R^{D},
(3) N(R^{A})SO₂R^{B},
(4) N(R^{A})SO₂N(R^{C})R^{D},
(5) N(R^{A})—C(═O)—C₁₋₆ alkylene-SO₂R^{B},
(6) N(R^{A})—C(═O)—C₁₋₆ alkylene-SO₂N(R^{C})R^{D},
(7) N(R^{A})C(═O)C(═O)N(R^{C})R^{D},
(8) N(R^{A})—C(═O)-HetA,
(9) N(R^{A})C(═O)C(═O)-HetA, or

R⁶ is —H or —C₁₋₆ alkyl;

n is an integer equal to 1 or 2;

each R^{A} is independently —H or —C₁₋₆ alkyl;

each R^{B} is independently —H or —C₁₋₆ alkyl;

R^{C} and R^{D} are each independently —H or —C₁₋₆ alkyl, or together with the nitrogen to which they are attached form a saturated 5- or 6-membered heterocyclic ring optionally containing a heteroatom in addition to the nitrogen attached to R^{C} and R^{D} selected from N, O, and S, where the S is optionally oxidized to S(O) or S(O)₂, and wherein the saturated heterocyclic ring is optionally substituted with 1 or 2 C₁₋₆ alkyl groups;

HetA is a 5- or 6-membered heteroaromatic ring containing from 1 to 4 heteroatoms independently selected from N, O and S, wherein the heteroaromatic ring is optionally substituted with 1 or 2 substituents each of which is independently —C₁₋₄ alkyl, —C₁₋₄ haloalkyl, —O—C₁₋₄ alkyl, —O—C₁₋₄ haloalkyl, or —CO₂R^{A} ; and

HetB is a 5- to 7-membered saturated heterocyclic ring containing from 1 to 4 heteroatoms independently selected from N, O and S, wherein each S is optionally oxidized to S(O) or S(O)₂, and the heterocyclic ring is optionally substituted with from 1 to 3 substituents each of which is independently halogen, —C₁₋₄ alkyl, —C₁₋₄ fluoroalkyl, —C(O)—C₁₋₄ alkyl, or —C₁₋₄ alkyl substituted with OH.

In an aspect of the preceding embodiment, in the compound of Formula I, R² is methyl; R³ is —H; and R⁴ is CH₂-phenyl wherein the phenyl is optionally substituted with 1 or 2 substituents each of which is independently bromo, chloro, fluoro, CH₃, CF₃, C(O)NH₂, C(O)NH(CH₃), C(O)N(CH₃)₂, SCH₃, SO₂CH₃, or SO₂N(CH₃)₂; and all other variables are as defined above. In a feature of this aspect, R⁴ is 4-fluorobenzyl, 3,4-dichlorobenzyl, 3-chloro-4-fluorobenzyl, or 4-fluoro-3-methylbenzyl.

In another feature of this aspect, R⁴ is 4-fluorobenzyl.

As used herein, the term “alkyl” refers to any linear or branched chain alkyl group having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any linear or branched chain alkylene group (or alternatively “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —CH₂)₁₋₂—, and —CH₂—. Also of interest is the alkylene —CH(CH₃)—.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “aryl” refers to (i) phenyl or (ii) a 9- or 10-membered bicyclic, fused carbocylic ring system in which at least one ring is aromatic. Aryl is typically phenyl or naphthyl, and is more typically phenyl.

The term “HetA” refers to an optionally substituted a 5- or 6-membered heteroaromatic ring containing from 1 to 4 heteroatoms independently selected from N, O and S. In one embodiment, HetA is an optionally substituted heteroaromatic ring selected from the group consisting of pyridinyl, pyrrolyl, pyrazinyl, pyrimidinyl, pyridazinyl, triazinyl, furanyl, thienyl, imidazolyl, pyrazolyl, triazolyl, tetrazolyl, oxazolyl, isooxazolyl, thiazoly, isothiazolyl, and oxadiazolyl; wherein the optional substitution is with 1 or 2 substituents each of which is independently —C₁₋₄ alkyl, —C₁₋₄ haloalkyl, —O—C₁₋₄ alkyl, —O—C₁₋₄ haloalkyl, or —CO₂—C₁₋₄ alkyl. It is understood that HetA can be attached to the rest of the compound of Formula I at any ring atom (i.e., any carbon atom or any heteroatom) provided that a stable compound results.

The term “HetB” refers to an optionally substituted a 5- to 7-membered saturated heterocyclic ring containing from 1 to 4 heteroatoms independently selected from N, O and S. In one embodiment, HetB is an optionally substituted saturated heterocyclic ring selected from the group consisting of pyrrolidinyl, imidazolidinyl, piperidinyl, piperazinyl, morpholinyl, thiomorpholinyl, thiazinanyl, and tetrahydropyranyl, wherein the optional substitution is with 1 or 2 substituents each of which is independently —C₁₋₄ alkyl, —C₁₋₄ haloalkyl, —C(O)CF₃, —C(O)CH₃, or —CH₂CH₂OH. It is understood that HetA can be attached to the rest of the compound of Formula I at any ring atom (i.e., any carbon atom or any heteroatom) provided that a stable compound results. In another embodiment, HetB is selected from the group consisting of 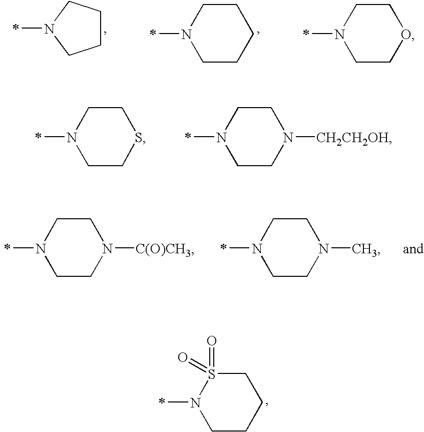
wherein* denotes the point of attachment to the rest of the molecule.

In the compound of Formula I, R^{C} and R^{D} together with the nitrogen to which they are attached can form a saturated 5- or 6-membered heterocyclic ring optionally containing a heteroatom in addition to the nitrogen attached to R^{C} and R^{D} selected from N, O, and S, where the S is optionally oxidized to S(O) or S(O)₂, and wherein the saturated heterocyclic ring is optionally substituted with 1 or 2 C₁₋₆ alkyl groups. In one embodiment, the saturated heterocyclic ring formed by R^{C} and R^{D} and the nitrogen to which they are attached is selected from the group consisting of 4-morpholinyl, 4-thiomorpholinyl, 1-piperidinyl, 1-piperazinyl optionally substituted with C₁₋₄ alkyl, and 1-pyrrolidinyl.

When any variable (e.g., R^{A} and R^{B}) occurs more than one time in Formula I or in any other formula depicting and describing a compound suitable for use in the present invention, its definition on each occurrence is independent of its definition at every other occurrence. Also, combinations of substituents and/or variables are permissible to the extent such combinations result in stable compounds.

A “stable” compound is a compound which can be prepared and isolated and whose structure and properties remain or can be caused to remain essentially unchanged for a period of time sufficient to allow use of the compound for the purposes described herein.

As a result of the selection of substituents and substituent patterns, certain of the compounds of Formula I whose salts can be employed in the present invention can have asymmetric centers and can occur as mixtures of stereoisomers, or as individual diastereomers, or enantiomers. The salts of all isomeric forms of these compounds, whether individually or in mixtures, can be employed in the present invention.

Compounds of Formula I can also exist as tautomers due to keto-enol tautomerism. The salts of all tautomers of the hydroxypyrimidinone compounds of Formula I, both singly and in mixtures, can be employed in the present invention.

Compounds embraced by Formula I are HIV integrase inhibitors. Representative compounds of Formula I other than those of Formula II are disclosed in WO 03/035077. Representative compounds of Formula I which are compounds of Formula II are disclosed in WO 2004/058757 and WO2004/058756.

A sixth embodiment of the present invention is the method for improving PK as originally set forth above or as set forth in the first or second embodiment, wherein the drug that is directly metabolized by UGT1A1 is Compound A, or a pharmaceutically acceptable salt thereof, wherein Compound A is N-(4-fluorobenzyl)-5-hydroxy-1-methyl-2-(1-methyl-1-{[(5-methyl-1,3,4-oxadiazol-2-yl)carbonyl]amino}ethyl)-6-oxo-1,6-dihydroxypyrimidine-4-carboxamide. The structure of Compound A is as follows:

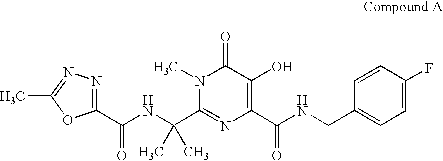

Compound A, which is disclosed in International Publication No. WO 03/035077, is a potent HIV integrase inhibitor.

Aspects of the sixth embodiment include the following, each of which is the method for improving PK as originally set forth in the sixth embodiment, and wherein:

(1) the amount of Compound A administered per day in the combination is in a range of from about 5 mg/kg to about 10 mg/kg of body weight and the amount of atazanavir administered per day in the combination is in a range of from about 2 mg/kg to about 10 mg/kg of body weight.

(2) the amount of Compound A administered per day is in a range of from about 5 mg/kg to about 10 mg/kg of body weight and the amount of atazanavir administered per day is in a range of from about 5 mg/kg to about 10 mg/kg.

(3) atazanavir is administered in the combination in an amount that, if administered alone, is less than that which is effective for treating HIV infection or AIDS.

(4) the amount of Compound A administered per day in the combination is in a range of from about 5 mg/kg to about 10 mg/kg of body weight and the amount of atazanavir administered per day in the combination is in a range of from about 2 mg/kg to about 5 mg/kg of body weight.

(5) the amount of Compound A administered per day in the combination is in a range of from about 5 mg/kg to about 10 mg/kg and the amount of atazanavir administered per day in the combination is less than 400 mg (e.g., from about 100 mg to about 350 mg per day, or from about 100 mg to about 250 mg per day, or from about 100 mg to about 200 mg per day).

(6) the amount of Compound A administered per day in the combination is in a range of from about 200 mg to about 1200 mg (e.g., from about 100 mg to about 600 mg twice per day) and the amount of atazanavir administered per day in the combination is less than 400 mg (e.g., from about 100 mg to about 350 mg per day, or from about 100 mg to about 250 mg per day, or from about 100 mg to about 200 mg per day).

It is understood that either or both Compound A and atazanavir can be alternatively employed in the above-described aspects of the sixth embodiment in the form of pharmaceutically acceptable salts. The references in these aspects to amounts of Compound A and atazanavir are to amounts of Compound A in its non-salt, free phenol form and to amounts of atazanavir in its non-salt, free base form.

A seventh embodiment of the present invention is the method for improving PK as originally set forth above or as set forth in either the first or second embodiment, wherein the drug that is directly metabolized by UGT1A1 is Compound A in the form of a potassium salt. Aspects of this embodiment include aspects analogous to aspects (1) to (6) set forth above for the sixth embodiment. In this embodiment and aspects thereof, the potassium salt of Compound A is preferably a crystalline potassium salt of Compound A, and is more preferably Form 1 crystalline potassium salt of Compound A, wherein the Form 1 K salt is an anhydrous crystalline salt characterized by an X-ray powder diffraction pattern obtained using copper K_{α} radiation (i.e., the radiation source is a combination of Cu K_{α1} and K_{α2} radiation) which comprises 2Θ values (i.e., reflections at 2Θ values) in degrees of 5.9,12.5, 20.0, 20.6 and 25.6.

An eighth embodiment of the present invention is the method for improving PK as originally set forth above or as set forth in either the first or second embodiment wherein the drug that is directly metabolized by UGT1A1 is a hydroxy polyhydro-2,6-naphthyridine dione compound of Formula III, or a pharmaceutically acceptable salt thereof: 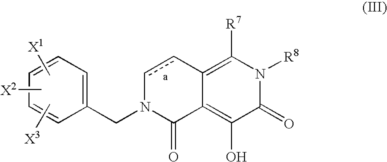
wherein:
bond [symbol] in the ring is a single bond or a double bond (e.g., is a single bond);

X¹ and X² are each independently:
(1) —H,
(2) —C₁₋₆ alkyl,
(3) —OH
(4) —O—C₁₋₆ alkyl,
(5) —C₁₋₆ haloalkyl,
(6) —O—C₁₋₆ haloalkyl,
(7) halogen,
(8) —CN,
(9) —N(R^{a})R^{b},
(10) —C(═O)N(R^{a})R^{b},
(11) —SR^{a},
(12) —S(O)R^{a},
(13) SO₂R^{a},
(14) —N(R^{a})SO₂R^{b},
(15) —N(R^{a})SO₂N(R^{a})R^{b},
(16) —N(R^{a})C(═O)R^{b},
(17) —N(R^{a})C(═O)—C(═O)N(R^{a})R^{b},
(18) —HetK,
(19) —C(═O)-HetK, or
(20) HetL;
   wherein each HetK is independently a C₄₋₅ azacycloalkyl or a C₃₋₄ diazacycloalkyl, either of which is optionally substituted with 1 or 2 substituents each of which is independently oxo or C₁₋₆ alkyl; and with the proviso that when HetK is attached to the rest of the compound via the —C(═O)— moiety, the HetK is attached to the —C(═O)— via a ring N atom; and
   each HetL is independently a 5- or 6-membered heteroaromatic ring containing from 1 to 4 heteroatoms independently selected from N, O and S, wherein the heteroaromatic ring is optionally substituted with from 1 to 4 substituents each of which is independently halogen, —C₁₋₆ alkyl, —C₁₋₆ haloalklyl, —O—C₁₋₆ alkyl, —O—C₁₋₆ haloalkyl, or hydroxy; or alternatively X¹ and X² are respectively located on adjacent carbons in the phenyl ring and together form methylenedioxy or ethylenedioxy;

X³ is:
(1) —H,
(2) —C₁₋₆ alkyl,
(3) —O—C₁₋₆ alkyl,
(4) —C₁₋₆ haloalkyl,
(5) —O—C₁₋₆ haloalkyl, or
(6) halogen;

R⁷ is:
(1) —C₁₋₆ alkyl,
(2) —CO₂R^{a},
(3) —C(═O)N(R^{a})R^{b},
(4) —C(═O)—N(R^{a})—(CH₂)₂₋₃—OR^{b},
(5) —N(R^{a})C(═O)R^{b},
(6) —N(R^{a})SO₂R^{b},
(7) —C₃₋₆ cycloalkyl, which is optionally substituted with from 1 to 4 substituents each of which is independently halogen, —C₁₋₆ alkyl, —CF₃, —O—C₁₋₆ alkyl, or —OCF₃,
(8) -HetK,
(9) —C(═O)-HetK,
(10) —C(═O)N(R^{a})-HetK,
(11) —C(═O)N(R^{a})—(CH₂)₀₋₂—(C₃₋₆ cycloalkyl), wherein the cycloalkyl is optionally substituted with from 1 to 4 substituents each of which is independently halogen, —C₁₋₆ alkyl, —CF₃, —O—C₁₋₆ alkyl, or —OCF₃, or
(12) —C(═O)N(R^{a})—CH₂-phenyl, wherein the phenyl is optionally substituted with from 1 to 4 substituents each of which is independently —C₁₋₆ alkyl, —O—C₁₋₆ alkyl, —CF₃, —OCF₃, or halogen;
(13) -HetL,
(14) —C(═O)N(R^{a})R^{c}, or
(15) halogen;
   wherein HetK is a 5- or 6-membered saturated heterocyclic ring containing a total of from 1 to 4 heteroatoms independently selected from 1 to 4 N atoms, from 0 to 2 O atoms, and from 0 to 2 S atoms, wherein the heterocyclic ring is optionally substituted with (i) from 1 to 4 substituents each of which is independently —C₁₋₆ alkyl, oxo, halogen, —C(═O)N(R^{a})R^{b}, —C(═O)C(═O)N(R^{a})R^{b}, —C(═O)R^{a}, —CO₂R^{a}, —SO₂R^{a}, or —SO₂N(R^{a})R^{b} and (ii) from zero to 1 C₃₋₆ cycloalkyl; and with the proviso that when HetK is attached to the rest of the compound via the —C(═O)— moiety, the HetK is attached to the —C(═O)— via a ring N atom;
   wherein HetL is a 5- or 6-membered heteroaromatic ring containing from 1 to 4 heteroatoms independently selected from N, O and S, wherein the heteroaromatic ring is optionally substituted with from 1 to 4 substituents each of which is independently —C₁₋₆ alkyl or —OH;

R⁸ is:
(1) —H,
(2) —C₁₋₆ alkyl,
(3) —C₃₋₆ cycloalkyl,
(4) —(CH₂)₁₋₂—C₃₋₆ cycloalkyl,
(5) —CH₂-phenyl wherein the phenyl is optionally substituted with from 1 to 4 substituents each of which is independently halogen, C₁₋₆ alkyl, C₁₋₆ haloalkyl, —O—C₁₋₆ alkyl, or —O—C₁₋₆ haloalkyl,
(6) —(CH₂)₁₋₂-HetM, wherein HetM is a 4- to 7-membered saturated heterocyclic ring containing from 1 to 2 heteroatoms independently selected from 1 to 2 N atoms, from zero to 1 O atom and from zero to 1 S atom, wherein the heterocyclic ring is attached to the rest of the molecule via a ring N atom, and the heterocyclic ring is optionally substituted with from 1 to 4 substituents each of which is independently —C₁₋₆ alkyl, —C₁₋₆ haloalkyl, —O—C₁₋₆ alkyl, —O—C₁₋₆ haloalkyl, oxo, —C(═O)N(R^{a})R^{b}, —C(═O)R^{a}, —CO₂R^{a}, —SO₂R^{a}, or —SO₂N(R^{a})R^{b},
(7) phenyl which is optionally substituted with from 1 to 4 substituents each of which is independently —C₁₋₆ alkyl, —O—C₁₋₆ alkyl, —C₁₋₆ haloalkyl, —O—C₁₋₆ haloalkyl, —OH, halogen, —CN, —NO₂, —C(═O)R^{a}, —CO₂R^{a}, —SO₂R^{a}, —N(R^{a})C(═O)—C₁₋₆ haloalkyl, —N(R^{a})C(═O)R^{b}, —N(R^{a})C(═O)N(R^{a})R^{b}, —N(R^{a})CO₂R^{b}, —N(R^{a})SO₂R^{b}, —C(═O)N(R^{d})R^{e}, or —SO₂N(R^{d})R^{e};
(8) a 5- or 6-membered heteroaromatic ring containing from 1 to 4 heteroatoms independently selected from N, O and S, wherein the heteroaromatic ring is optionally substituted with from 1 to 4 substituents each of which is independently —C₁₋₆ alkyl, —C₁₋₆ haloalkyl, —O—C₁₋₆ alkyl, —O—C₁₋₆ haloalkyl, or —OH,
(9) C₁₋₆ alkyl substituted with —O—C₁₋₆ alkyl, —CN, —N(R^{a})R^{b}, —C(═O)N(R^{a})R^{b}, —C(═O)R^{a}, —CO₂R^{a}, —SO₂R^{a}, or —SO₂N(R^{a})R^{b}, or
(10) —C₁₋₆ haloalkyl;

each R^{a} is independently H or C₁₋₆ alkyl;

each R^{b} is independently H or C₁₋₆ alkyl;

R^{c} is C₁₋₆ haloalkyl or C₁₋₆ alkyl substituted with —CO₂R^{a}, —SO₂R^{a}, —SO₂N(R^{a})R^{b}, or N(R^{a})R^{b}; and

each R^{d} and R^{e} are independently H or C₁₋₆ alkyl, or together with the N atom to which they are attached form a 4- to 7-membered saturated heterocyclic ring optionally containing a heteroatom in addition to the nitrogen attached to R^{d} and R^{e} selected from N, O, and S, wherein the S is optionally oxidized to S(O) or S(O)₂, and wherein the saturated heterocyclic ring is optionally substituted with from 1 to 4 substituents each of which is independently halogen, —CN, —C₁₋₆ alkyl, —OH, oxo, —O—C₁₋₆ alkyl, —C₁₋₆ haloalkyl, —C(═O)R^{a}, —CO₂R^{a}, —SO₂R^{a}, or —SO₂N(R^{a})R^{b}.

A ninth embodiment of the present invention is the method for improving PK as originally set forth above or as set forth in either the first or second embodiment wherein the drug that is directly metabolized by UGT1A1 is a hydroxy polyhydro-2,6-naphthyridine dione compound of Formula IV, or a pharmaceutically acceptable salt thereof: 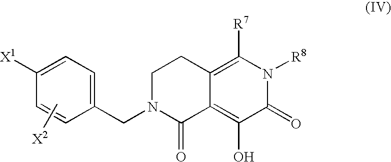
wherein:

X¹ is: (1) —H, (2) bromo, (3) chloro, (4) fluoro, or (5) methoxy;

X² is: (1) —H, (2) bromo, (3) chloro, (4) fluoro, (5) methoxy, (6) —C₁₋₄ alkyl, (7) —CF₃, (8) —OCF₃, (9) —CN, or (10) —SO₂(C₁₋₄ alkyl);

R⁷ is: (1) —CO₂H, (2) —C(═O)—O—C₁₋₄ alkyl, (3) —C(═O)NH₂, (4) —C(═O)NH—C₁₋₄ alkyl, (5) —C(═O)N(C₁₋₄ alkyl)₂, (6) —C(═O)—NH—(CH₂)₂₋₃—O—C₁₋₄ alkyl, (7) —C(═O)—N(C₁₋₄ alkyl)-(CH₂)₂₋₃—O—C₁₋₄ alkyl, (8) —NHC(═O)—C₁₋₄ alkyl, (9) —N(C₁₋₄ alkyl)C(═O)—C₁₋₄ alkyl, (10) —NHSO₂—C₁₋₄ alkyl, (11) —N(C₁₋₄ alkyl)SO₂—C₁₋₄ alkyl, (12) —C(═O)-HetK, wherein HetK is: 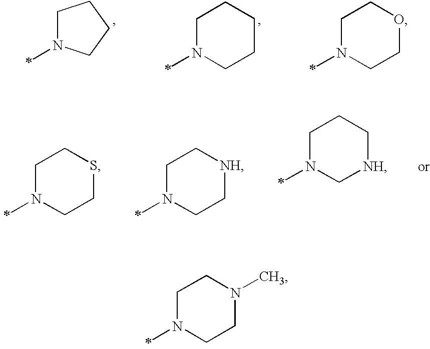
wherein the asterisk * denotes the point of attachment to the rest of the compound, (13) —C(═O)NH—(CH₂)₀₋₁—(C₃₋₆ cycloalkyl), (14) —C(═O)N(C₁₋₄ alkyl)-(CH₂)₀₋₁—(C₃₋₆ cycloalkyl), (15) —C(═O)NH—CH₂-phenyl, or (16) —C(═O)N(C₁₋₄ alkyl)—CH₂-phenyl; and

R⁸ is: (1) —H, (2) —C₁₋₄ alkyl, (3) cyclopropyl, (4) cyclobutyl, (5) —CH₂-cyclopropyl, (6) —CH₂-cyclobutyl, or (7) —CH₂-phenyl.

In an aspect of the ninth embodiment, X¹ is fluoro; X² is —H or chloro; R⁷ is:
(1) —C(═O)N(C₁₋₃ alkyl)₂,
(2) —C(═O)-HetK, wherein HetK is: 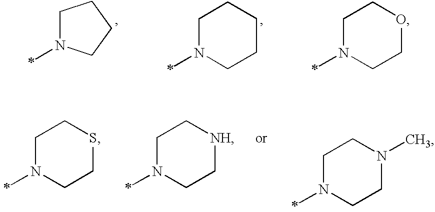
   wherein the asterisk * denotes the point of attachment to the rest of the compound,
(3) —C(═O)N(C₁₋₃ alkyl)-(CH₂)₀₋₁-cyclopropyl, or
(4) —C(═O)N(C₁₋₃ alkyl)-(CH₂)₀₋₁-cyclobutyl; and R⁸ is —C₁₋₄ alkyl.

A tenth embodiment of the present invention is the method for improving PK as originally set forth above or as set forth in either the first or second embodiment wherein the drug that is directly metabolized by UGT1A1 is selected from the group consisting of: 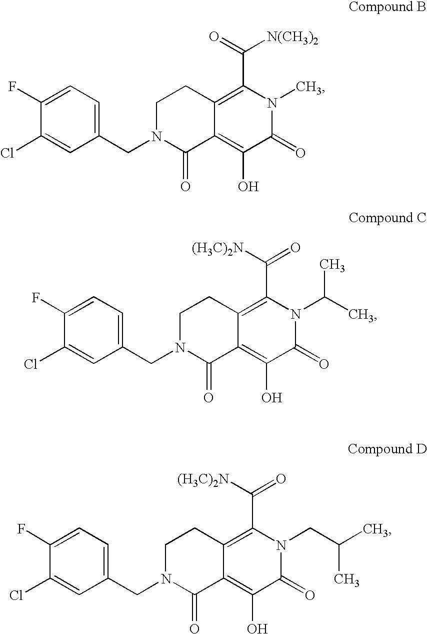
and pharmaceutically acceptable salts thereof.

In an aspect of the tenth embodiment, the compound is Compound B. In another aspect of the tenth embodiment, the compound is Compound C. In still another aspect of the tenth embodiment, the compound is Compound D.

Compounds embraced by Formula III and Formula IV and Compounds B, C and D are HIV integrase inhibitors. These compounds and their preparation and use are further described in WO 2005/087768.

The present invention also includes a method for improving circulation level of an orally administered drug that is directly metabolized by UGT1A1 which comprises orally administering to a mammal in need of treatment with the drug an effective amount of a combination of the drug or a pharmaceutically acceptable salt thereof and atazanavir or a pharmaceutically acceptable salt thereof. An improvement in the circulation level of a drug means herein an increase in the level of drug in the systemic circulation (e.g., the bloodstream of a human being) compared to the corresponding value obtained by administration of the drug in the absence of atazanavir. Embodiments of this method include the following, each of which is the method for improving circulation level as just set forth, and wherein:

(1) atazanavir is administered in the combination in an amount sufficient to improve the circulation level of the drug by at least about 10% with respect to the circulation level of the drug administered in the absence of atazanavir.

(2) the mammal in need of treatment with the drug is a human.

(3) the mammal in need of treatment with the drug is a human, and the drug that is directly metabolized by UGT1A1 is selected from the group consisting of ezetimibe, raloxifene, estradiol, and pharmaceutically acceptable salts thereof.

(4) the drug that is directly metabolized by UGT1A1 is an HIV integrase inhibitor.

(4a) the method is as set forth in (4), wherein the mammal in need of treatment with the drug is a human.

(4b) the method is as set forth in (4), wherein atazanavir is administered in the combination in an amount sufficient to improve the circulation level of the integrase inhibitor by at least about 10% with respect to the circulation level of Compound I administered in the absence of atazanavir.

(4c) the method is as set forth in (4), wherein atazanavir is administered in the combination in an amount that, if administered alone, is less than that which is effective for treating HIV infection or AIDS.

(4d) the method is as set forth in (4), wherein the method incorporates feature (4a) and either feature (4b) or (4c).

(4e) the method is as set forth in (4), wherein the method incorporates features (4a), (4b) and (4c).

(5) the drug that is directly metabolized by UGT1A1 is a compound of Formula I as heretofore defined, or a pharmaceutically acceptable salt thereof.

(5a) the method is as set forth in (5), wherein the mammal in need of treatment with the drug is a human.

(5b) the method is as set forth in (5), wherein atazanavir is administered in the combination in an amount sufficient to improve the circulation level of Compound I by at least about 10% with respect to the circulation level of Compound I administered in the absence of atazanavir.

(5c) the method is as set forth in (5), wherein atazanavir is administered in the combination in an amount that, if administered alone, is less than that which is effective for treating HIV infection or AIDS.

(5d) the method is as set forth in (5), wherein the method incorporates feature (5a) and either feature (5b) or (5c).

(5e) the method is as set forth in (5), wherein the method incorporates features (5a), (5b) and (5c).

(6) the drug that is directly metabolized by UGT1A1 is Compound A as heretofore defined, or a pharmaceutically acceptable salt thereof.

(6a) the method is as set forth in (6), wherein the mammal in need of treatment with the drug is a human.

(6b) the method is as set forth in (6), wherein atazanavir is administered in the combination in an amount sufficient to improve the circulation level of Compound A by at least about 10% with respect to the circulation level of Compound A administered in the absence of atazanavir.

(6c) the method is as set forth in (6), wherein the amount of Compound A administered per day in the combination is in a range of from about 5 mg/kg to about 10 mg/kg of body weight and the amount of atazanavir administered per day in the combination is in a range of from about 2 mg/kg to about 10 mg/kg (or from about 5 mg/kg to about 10 mg/kg) of body weight.

(6d) the method is as set forth in (6), wherein atazanavir is administered in the combination in an amount that, if administered alone, is less than that which is effective for treating HIV infection or AIDS.

(6e) the method is as set forth in (6), wherein the amount of Compound A administered per day in the combination is in a range of from about 5 mg/kg to about 10 mg/kg of body weight and the amount of atazanavir administered per day in the combination is in a range of from about 2 mg/kg to about 5 mg/kg of body weight.

(6f) the method is as set forth in (6), wherein the amount of Compound A administered per day in the combination is in a range of from about 5 mg/kg to about 10 mg/kg of body weight and the amount of atazanavir administered per day in the combination is less than 400 mg (e.g., from about 100 mg to about 350 mg per day, or from about 100 mg to about 250 mg per day, or from about 100 mg to about 200 mg per day).

(6g) the method is as set forth in (6), wherein the amount of Compound A administered per day in the combination is in a range of from about 200 mg to about 1200 mg (e.g., from about 100 mg to about 600 mg twice per day) and the amount of atazanavir administered per day in the combination is less than 400 mg (e.g., from about 100 mg to about 350 mg per day, or from about 100 mg to about 250 mg per day, or from about 100 mg to about 200 mg per day).

(6h) the method is as set forth in (6), wherein the method incorporates feature (6a) and any one of features (6b) to (6g).

(7) the drug that is directly metabolized by UGT1A1 is a potassium salt of Compound A (preferably a crystalline potassium salt of Compound A, and more preferably Form 1 crystalline potassium salt of Compound A).

(7a) to (7h) each of the methods is as set forth in (7), wherein each method respectively incorporates features analogous to features (6a) to (6h) set forth above.

(8) the drug that is directly metabolized by UGT1A1 is a compound of Formula III as heretofore defined, or a pharmaceutically acceptable salt thereof.

(8a) to (8e) each of the methods is as set forth in (8), wherein each method respectively incorporates features analogous to features (5a) to (5e) set forth above.

(9) the drug that is directly metabolized by UGT1A1 is a compound of Formula IV as heretofore defined, or a pharmaceutically acceptable salt thereof.

(9a) to (9e) each of the methods is as set forth in (9), wherein each method respectively incorporates features analogous to features (5a) to (5e) set forth above.

(10) the drug that is directly metabolized by UGT1A1 is a compound selected from the group consisting of Compound B, Compound C and Compound D, or a pharmaceutically acceptable salt thereof.

(10a) to (10e) each of the methods is as set forth in (10), wherein each method respectively incorporates features analogous to features (5a) to (5e) set forth above.

The present invention also includes a method for inhibiting HIV integrase which comprises administering to a mammal in need of such inhibition an effective amount of a combination of an HIV integrase inhibitor that is directly metabolized by UGT1A1 or a pharmaceutically acceptable salt thereof and atazanavir or a pharmaceutically acceptable salt thereof. Embodiments of this method include the following, each of which is the method for inhibiting HIV integrase as just set forth and wherein:

(1) atazanavir is administered in the combination in an amount sufficient to improve the PK of the HIV integrase inhibitor by at least about 10% with respect to the PK of the HWV integrase inhibitor administered in the absence of atazanavir.

(2) the mammal in need of treatment with the HIV integrase inhibitor is a human.

(3) the HIV integrase inhibitor that is directly metabolized by UGT1A1 is a compound of Formula I as heretofore defined, or a pharmaceutically acceptable salt thereof.

(3a) the method is as set forth in (3), wherein the mammal in need of treatment with the drug is a human.

(3b) the method is as set forth in (3), wherein atazanavir is administered in the combination in an amount sufficient to improve the PK of the HIV integrase inhibitor by at least about 10% with respect to the PK of the HIV integrase inhibitor administered in the absence of atazanavir.

(3c) the method is as set forth in (3), wherein atazanavir is administered in the combination in an amount that, if administered alone, is less than that which is effective for treating HIV infection or AIDS.

(3d) the method is as set forth in (3), wherein the method incorporates feature (3a) and either or both features (3b) and (3c).

(4) the HIV integrase inhibitor that is directly metabolized by UGT1A1 is Compound A as heretofore defined, or a pharmaceutically acceptable salt thereof.

(4a) the method is as set forth in (4), wherein the mammal in need of treatment with the drug is a human.

(4b) the method is as set forth in (4), wherein atazanavir is administered in the combination in an amount sufficient to improve the PK of Compound A by at least about 10% with respect to the PK of Compound A administered in the absence of atazanavir.

(4c) the method is as set forth in (4), wherein the amount of Compound A administered per day in the combination is in a range of from about 5 mg/kg to about 10 mg/kg of body weight and the amount of atazanavir administered per day in the combination is in a range of from about 2 mg/kg to about 10 mg/kg (or from about 5 mg/kg to about 10 mg/kg) of body weight.

(4d) the method is as set forth in (4), wherein atazanavir is administered in the combination in an amount that, if administered alone, is less than that which is effective for treating HIV infection or AIDS.

(4e) the method is as set forth in (4), wherein the amount of Compound A administered per day in the combination is in a range of from about 5 mg/kg to about 10 mg/kg of body weight and the amount of atazanavir administered per day in the combination is in a range of from about 2 mg/kg to about 5 mg/kg of body weight.

(4f) the method is as set forth in (4), wherein the amount of Compound A administered per day in the combination is in a range of from about 5 mg/kg to about 10 mg/kg of body weight and the amount of atazanavir administered per day in the combination is less than 400 mg (e.g., from about 100 mg to about 350 mg per day, or from about 100 mg to about 250 mg per day, or from about 100 mg to about 200 mg per day).

(4g) the method is as set forth in (4), wherein the amount of Compound A administered per day in the combination is in a range of from about 200 mg to about 1200 mg (e.g., from about 100 mg to about 600 mg twice per day) and the amount of atazanavir administered per day in the combination is less than 400 mg (e.g., from about 100 mg to about 350 mg per day, or from about 100 mg to about 250 mg per day, or from about 100 mg to about 200 mg per day).

(4h) the method is as set forth in (4), wherein the method incorporates feature (4a) and any one of features (4b) to (4g).

(5) the HIV integrase inhibitor that is directly metabolized by UGT1A1 is a potassium salt of Compound A (preferably a crystalline potassium salt of Compound A, and more preferably Form 1 crystalline potassium salt of Compound A).

(5a) to (5h) each of the methods is as set forth in (5), wherein each method respectively incorporates features analogous to features (4a) to (4h) set forth above.

(6) the HIV integrase inhibitor that is directly metabolized by UGT1A1 is a compound of Formula III, or a pharmaceutically acceptable salt thereof.

(6a) to (6d) each of the methods is as set forth in (6), wherein each method respectively incorporates features analogous to features (3a) to (3d) set forth above.

(7) the HIV integrase inhibitor that is directly metabolized by UGT1A1 is a compound of Formula IV, or a pharmaceutically acceptable salt thereof.

(7a) to (7d) each of the methods is as set forth in (7), wherein each method respectively incorporates features analogous to features (3a) to (3d) set forth above.

(8) the HIV integrase inhibitor that is directly metabolized by UGT1A1 is a compound selected from the group consisting of Compound B, Compound C and Compound D, or a pharmaceutically acceptable salt thereof.

(8a) to (8d) each of the methods is as set forth in (8), wherein each method respectively incorporates features analogous to features (3a) to (3d) set forth above.

The present invention also includes a method for treating HIV infection or AIDS, for prophylaxis of HIV infection or AIDS, or for delaying the onset of AIDS which comprises orally administering to a mammal in need of such treatment, prophylaxis, or delay an effective amount of a combination of an HIV integrase inhibitor that is directly metabolized by UGT1A1 or a pharmaceutically acceptable salt thereof and atazanavir or a pharmaceutically acceptable salt thereof. Embodiments of this method include embodiments analogous to embodiments (1), (2), (3) to (3d), (4) to (4h), (5) to (5h), (6) to (6d), (7) to (7d) and (8) to (8d) set forth above for the method for inhibiting HIV integrase.

The present invention also includes a pharmaceutical combination for oral administration to a mammal comprising a drug that is useful for the treatment or prophylaxis of a disease or condition and that is directly metabolized by UGT1A1, or a pharmaceutically acceptable salt thereof, and atazanavir or a pharmaceutically acceptable salt thereof, wherein the drug and atazanavir are each employed in an amount that provides therapeutic or prophylactic efficacy of the drug. Embodiments of the combination include the following, each of which is the combination as just described and wherein:

(1) the mammal to which the combination is administered is a human.

(2) the atazanavir is administered in the combination in an amount sufficient to improve the pharmacokinetics of the drug by at least about 10% with respect to the phairmacokinetics of the drug administered in the absence of atazanavir.

(3) the mammal to which the combination is administered is a human, and the drug is selected from the group consisting of ezetimibe, raloxifene, estradiol, and pharmaceutically acceptable salts thereof.

(4) the combination is a single pharmaceutical composition which further comprises a pharmaceutically acceptable carrier.

(5) the combination incorporates feature (1) and either or both features (2) and (4).

(6) the combination incorporates feature (2) and either or both features (3) and (4).

(7) the combination incorporates features (3) and (4).

The present invention also includes a pharmaceutical combination for oral administration to a mammal comprising an HIV integrase inhibitor that is directly metabolized by UGT1A1 or a pharmaceutically acceptable salt thereof and atazanavir or a pharmaceutically acceptable salt thereof, wherein the HIV integrase inhibitor and atazanavir are each employed in an amount that provides efficacy of the integrase inhibitor for (i) treatment of HIV infection or AIDS, (ii) prophylaxis of HIV infection or AIDS; or (iii) inhibition of HIV integrase. Embodiments of this combination include the combinations recited in embodiments (1), (2), (3) to (3d), (4) to (4h), (5) to (5h), (6) to (6d), (7) to (7d) and (8) to (8d) set forth above for the method for inhibiting HIV integrase. Further embodiments of this combination include the combination as originally set forth and as set forth in each of the foregoing embodiments, wherein the combination is a single pharmaceutical composition which further comprises a pharmaceutically acceptable carrier.

The present invention also includes use of atazanavir, or a pharmaceutically acceptable salt thereof, in combination with an orally administered drug that is directly metabolized by UGT1A1, or a pharmaceutically acceptable salt thereof, for improving the pharmacokinetics (or the circulation level) of the drug in a mammal in need of treatment with the drug. The present invention further includes the use of atazanavir, or a pharmaceutically acceptable salt thereof, in combination with an orally administered drug that is directly metabolized by UGT1A1, or a pharmaceutically acceptable salt thereof, in the manufacture of a medicament for improving the pharmacokinetics (or the circulation level) of the drug in a mammal in need of treatment with the drug. Embodiments of these uses are analogous to the embodiments set forth above for the corresponding method claims.

The present invention further includes use of atazanavir, or a pharmaceutically acceptable salt thereof, in combination with an orally administered HIV integrase inhibitor, that is directly metabolized by UGT1A1, or a pharmaceutically acceptable salt thereof, for inhibiting HIV integrase in a mammal in need of such inhibition. The present invention also includes use of atazanavir, or a pharmaceutically acceptable salt thereof, in combination with an orally administered HIV integrase inhibitor that is directly metabolized by UGT1A1, or a pharmaceutically acceptable salt thereof, in the manufacture of a medicament for inhibiting HIV integrase in a mammal in need of such inhibition. Embodiments of these uses are analogous to the embodiments set forth above for the corresponding method claims.

The present invention further includes use of atazanavir, or a pharmaceutically acceptable salt thereof, in combination with an orally administered HIV integrase inhibitor that is directly metabolized by UGT1A1, or a pharmaceutically acceptable salt thereof, for treating HIV infection or ADDS, for prophylaxis of HIV infection or AIDS, or for delaying the onset of AIDS in a mammal in need of such treatment, prophylaxis, or delay. The present invention also includes use of atazanavir, or a pharmaceutically acceptable salt thereof, in combination with an orally administered HIV integrase inhibitor that is directly metabolized by UGT1A1, or a pharmaceutically acceptable salt thereof, in the manufacture of a medicament for treating HIV infection or AIDS, for prophylaxis of HIV infection or AIDS, or for delaying the onset of AIDS in a mammal in need of such treatment, prophylaxis, or delay. Embodiments of these uses are analogous to the embodiments set forth above for the corresponding method claims.

The combination of atazanavir and the drug directly metabolized by UGT1A1, whether as a single composition or as separate compositions, are administered orally. Liquid compositions can be employed including, for example, pharmaceutically acceptable emulsions, solutions, suspensions, syrups, and elixirs. These liquid compositions can be prepared according to techniques known in the art and can employ any of the usual media such as water, glycols, oils, alcohols and the like. Solid compositions can also be employed including, for example, powders, granules, pills, capsules and tablets. The solid compositions can be prepared according to techniques known in the art and can employ such solid excipients as starches, sugars, kaolin, lubricants, binders, disintegrating agents and the like.

The daily dose of atazanavir to be administered to a human or other mammal in combination with the UGT1A1-metabolized drug is typically an amount sufficient to improve the pharmacokinetics of the drug by at least about 10% with respect to the pharmacokinetics of the drug administered in the absence of atazanavir. Guidance for establishing a suitable oral dose of atazanavir can be found in U.S. Pat. No. 5,849,911 and in the label for the approved drug product REYATAZ™ (atazanavir sulfate capsules; see, e.g., Physicians' Desk Reference, 2004 edition, pp. 1080-1088). The daily oral dose of the drug metabolized by UGT1A1 to be administered in combination with atazanavir is an amount which is effective against the particular disease or condition being treated or prevented. Guidance for establishing the appropriate daily dose for such drugs is known in the art. Guidance for many drugs can be found, for example, in the 2004 edition of the Physicians' Desk Reference. Dosing levels can also be found in the patent literature; e.g., information on dosage levels for ezetimibe and raloxifene can be found in U.S. Pat. No. RE37721 and U.S. Pat. No. 6,458,811 respectively. The specific dose levels of atazanavir and the drug will depend upon a variety of factors including (i) the activity of the particular drug employed in the combination; (ii) the age, body weight, general health, sex, and diet of the subject (human or other mammal); (iii) the mode of oral administration, (iv) the rate of excretion, and (v) the severity of the particular disease or condition being treated. The person of ordinary skill in the art can determine the appropriate oral doses of atazanavir and the drug for the treatment or prophylaxis of a particular disease or condition in a particular subject (i.e., human or other mammal) without undue experimentation.

Compounds embraced by Formula I, Formula III and Formula IV can be administered in a dosage range of from about 0.001 to about 1000 mg/kg of mammal (e.g., human) body weight per day in a single dose or in divided doses. One preferred dosage range is from about 0.01 to about 500 mg/kg body weight per day in a single dose or in divided doses. Another preferred dosage range is from about 0.1 to about 100 mg/kg body weight per day in single or divided doses. Compositions containing a compound of Formula I can suitably be provided in the form of tablets or capsules for oral administration, wherein each tablet or capsule contains from about 1 to about 1000 milligrams of the active ingredient, particularly 1, 5, 10, 15, 20, 25, 50, 75, 100, 150, 200, 250, 300, 400, 500, 600, 700, 800, 900 and 1000 milligrams of the active ingredient for the symptomatic adjustment of the dosage to the patient to be treated. Of course, the specific dose level and frequency of dosage for any particular patient can vary and will depend upon a variety of factors including factors (i) to (v) set forth in the preceding paragraph.

Suitable total daily doses of Compound A and atazanavir include the following:

| Compound A | atazanavir |
|---|---|
| about 5 mg/kg to about 10 mg/kg | about 2 mg/kg to about 10 mg/kg |
| about 5 mg/kg to about 10 mg/kg | about 5 mg/kg to about 10 mg/kg |
| about 5 mg/kg to about 10 mg/kg | about 2 mg/kg to about 5 mg/kg |
| (adult human) | less than 400 mg |
| about 5 mg/kg to about 10 mg/kg | (e.g., about 100 mg to about 350 mg, |
| | about 100 mg to about 250 mg, or |
| | about 100 mg to about 200 mg) |
| (adult human) | less than 400 mg |
| about 200 mg to about 1200 mg | (e.g., about 100 mg to about 350 mg, |
| (e.g., about 100 mg to about | about 100 mg to about 250 mg, or |
| 600 mg given twice daily) | about 100 mg to about 200 mg) |
| (adult human): about 800 mg | less than 400 mg |
| (e.g., about 400 mg given twice | (e.g., about 100 mg to about 350 mg, |
| daily) | about 100 mg to about 250 mg, or |
| | about 100 mg to about 200 mg) |

Compound A is preferably dosed in the form of a potassium salt (especially the Form 1 crystalline K salt). In a preferred embodiment, the potassium salt of Compound A is administered orally in a pharmaceutical composition comprising the Compound A K salt and hydroxypropylmethylcellulose (e.g., HPMC 2910), wherein the composition is compressed into a tablet. In another preferred embodiment, the potassium salt of Compound A is administered orally in a pharmaceutical composition comprising the Compound A K salt, a poloxamer (e.g., poloxamer 407), hydroxypropylmethylcellulose (e.g., BPMC K4M), and lactose (e.g., lactose hydrous spray dried), wherein the composition is compressed into a tablet.

Unless expressly stated to the contrary, all ranges cited herein are inclusive. For example, a heterocyclic ring described as containing from “1 to 4 heteroatoms” means the ring can contain 1, 2, 3 or 4 heteroatoms. As another example, a daily dose of Compound A of from about 5 mg/kg to about 10 mg/kg of body weight means the dose can be about 5 mg/kg, or about 10 mg/kg, or any value in between.

Abbreviations used herein include the following: ACN=acetonitrile; AIDS=acquired immunodeficiency syndrome; ARC=AIDS related complex; Bz=benzoyl; CBz=butyloxycarbonyl; DEA=diisopropylethylamine; DMADC=dimethylacetylene dicarboxylate; DMF=N,N-dimethylformamide; DMSO=dimethylsulfoxide; DSC=differential scanning calorimetry; EDTA=ethylenediaminetetraacetic acid; Eq.=equivalent(s); EtOH=ethanol; HIV=human immunodeficiency virus; HPLC=high-performance liquid chromatography; HPMC=hydroxypropylmethylcellulose; IPA=isopropyl alcohol; KF=Karl Fisher titration for water; LC=liquid chromatography; LCAP=LC area percent; LCWP=LC weight percent; Me=methyl; MeOH=methanol; MS=mass spectroscopy; MSA=methanesulfonic acid; MTBE=methyl tertiary butyl ether; MW=molecular weight; NMM=N-methylmorpholine; NMR=nuclear magnetic resonance; PK=pharmacokinetics; TG=thermogravimetric; THF=tetrahydrofuran; UDPGA=uridine 5′-diphospho-glucuronic acid; XRPD=x-ray powder diffraction.

The following examples serve only to illustrate the invention and its practice. The examples are not to be construed as limitations on the scope or spirit of the invention. Compound B in Examples 4 and 5 is 5-(1,1-dioxido-1,2-thiazinan-2-yl)-N-(4-fluorobenzyl)-8-hydroxy-1,6-naphthyridine-7-carboxamide, which is disclosed in US 2003/055071.

### EXAMPLE 1

Preparation of Compound A and a Crystalline Potassium Salt Thereof

Step 1: Strecker Amine Formation

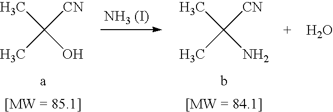

| | | | | | | density |
|---|---|---|---|---|---|---|
| Material | MW | Eq. | Moles | Mass | Volume | (g/mL) |
| acetone cyanohydrin | 85.1 | 1.0 | 129.3 | 11.0 kg | 11.8 L | 0.932 |
| (a) | | | | | | |
| MTBE | | 4.0 | | | 44 L | |
| ammonia (g) | 17.03 | 1.5 | 193.9 | 3.30 kg | 4.9 L | 0.674 |

Acetone cyanohydrin (11.5 kg, 12.3 L) was charged to a 5-gallon autoclave and the vessel placed under 5 psi nitrogen pressure. The autoclave was cooled to 10° C., and ammonia gas (˜3.44 kg), pressurized to 30 psi, was fed into the vessel until the reaction reached complete conversion as determined by GC assay (less than 0.5% a). The resulting suspension was transferred to a polyjug and the autoclave rinsed with MTBE (approximately 17 L). The reaction mixture and rinse were then charged to a 100-L extractor followed by MTBE (15 L), the mixture agitated, and the layers carefully separated. The aqueous layer was back-extracted with MTBE (5 L) and the layers carefully separated. The organic layers were combined and charged to a 100 L flask, equipped with a batch concentrator, through an in-line filter, and the batch was concentrated (15-20° C., low vacuum) to about 20 L to remove any excess ammonia. The aminonitrile was obtained in 97% assay yield (11.1 kg) by NMR as a solution in MTBE.

Step 2: Addition of Benzyloxycarbonyl (CBz) Protective Group

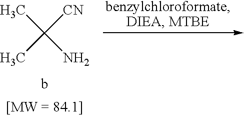

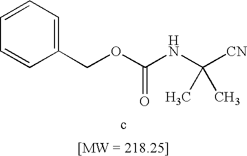

| Material | MW | Eq. | Moles | Mass | Volume |
|---|---|---|---|---|---|
| aminonitrile (b) | 84.1 | | 52.85 | 4.44 assay kg | |
| benzylchloroformate | 170.6 | 1.2 | 63.4 | 10.8 kg | |
| DIEA | 129.25 | 1.3 | 68.7 | 8.88 | |
| MTBE | | | | | 62.5 L |

To a visually clean 100-L flask containing a 5-L addition funnel, thermocouple and nitrogen inlet was charged a 59 wt. % solution of cyanoamine b in MTBE (4.44 assay kg). The solution was further diluted with MTBE (62.5 L) to bring the concentration to approximately 15 mL/g. Benzylchloroformate (1.20 equiv, 10.42 kg, 61.10 mol) was then charged in over 15 minutes via the addition funnel at such a rate as to maintain the batch temperature below 35° C. DIEA (1.3 equiv, 8.88 kg, 68.70 mol) was then added over 1.5 hours to the yellow slurry while maintaining the batch temperature below 35° C. The slurry became slightly more soluble as DEA was added but two phases were observed when stirring was stopped. The reaction mixture was aged for 16 hours at 20-25° C., after which DI water (20 L, 4.5 mL/g) was charged into the batch. The batch was then transferred to a 100-L extractor and the phases were separated. The organic layer was then washed with 3×10 L of water and then 15 L of brine. The organic layer was transferred via a 10 μm inline filter to a 100 L round bottom flask and subsequently solvent switched to 90:10 heptane:MTBE. Crystallization occurred during the solvent switch and the resulting white crystalline product was filtered and washed with 3×5 L of 90:10 heptane:MTBE. A total of 10.1 kg of product (88% yield) was obtained in greater than 99 HPLC A %. A total of 26.7 kg of product was obtained in 3 batches with an average isolated yield of 86%.

Step 3: Amidoxime Formation

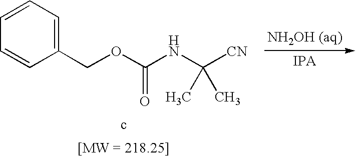

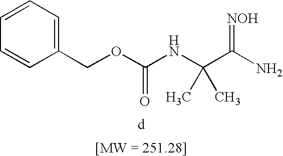

| Material | MW | Eq. | Mass | Volume |
|---|---|---|---|---|
| protected aminonitrile (c) | 218.25 | 1 | 15 g | |
| NH₂OH (50 wt. % in water) | | 1.2 | | 5.05 mL |
| IPA | | | 40 mL+ | |
| | | | 10 mL | |
| n-heptane | | | 40 mL+ | |
| | | | 50 mL | |

A solution of aminonitrile (15 g) in IPA (40 mL) was warmed to 60° C. with stirring and NH₂OH in water (5.05 mL) was added at this temperature over the course of 20 minutes. The clear mixture was then aged at 60° C. for 3 hours, wherein product began to crystallize out of solution at this temperature after 2 hours. The slurry was then cooled to 0°-5° C. and n-heptane (40 mL) was added dropwise over 20 minutes. After stirring for 2 hours at 0°-5° C., the slurry was filtered and the cake was washed with a 20% EPA in heptane solution (60 mL), and then dried under vacuum with a nitrogen stream at room temperature to give pure amide oxime in 88% yield.

Step 4: Formation of Hydroxypyrimidinone

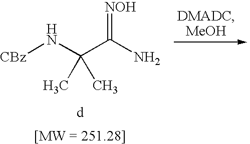

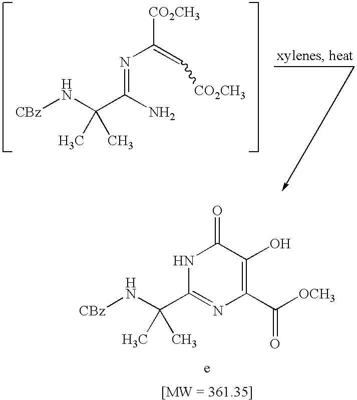

| | | | | | Density |
|---|---|---|---|---|---|
| Material | MW | Eq. | Mass | Volume | (g/mL) |
| amidoxime (d) | 251.28 | 1 | 2.9 kg | | |
| DMADC | 142.11 | 1.08 | 1.77 | | 1.16 |
| MeOH | | | | 12 L + 6 L | |
| xylenes | | | | 15 L | |
| MTBE | | | | 9 L | |

To a slurry of amidoxime (2.90 kg) in methanol (12 L) was added dimethyl acetylenedicarboxylate (1.77 kg) over 20 minutes. A slow exotherm ensued such that the temperature of the slurry increased from 20° C. to 30° C. over 15-20 minutes. After 1.5 hours, HPLC indicated greater than 95% conversion to the intermediate cis/trans adducts. The solvent was then switched to xylenes under reduced pressure (maximum temperature=50° C.), wherein 2 volumes [2×7.5 L] were added and reduced to a final volume of 7.5 L. The reaction mixture was then heated to 90° C. and kept at this temperature for 2 hours, while flushing the remaining MeOH out with a nitrogen sweep. The temperature was then increased in 10° C. increments over 3.5 hours to 125° C. and held at this temperature for 2 hours. The temperature was then finally increased to 135° C. for 5 hours. The reaction mixture was then cooled to 60° C. and MeOH (2.5 L) was added. After 30 minutes MTBE (9 L) was added slowly to build a seed bed. The batch was then cooled to 0° C. for 14 hours, and then further cooled to −5° C. and aged 1 hour before filtration. The solids were displacement washed with 10% MeOH/MTBE (6 L then 4 L; pre-chilled to 0° C.) and dried on the filter pot under a nitrogen sweep to afford 2.17 kg (51.7% corrected yield; 99.5 wt %).

HPLC method: Column: Zorbax C-8 4.6 mm×250 mm, 40% ACN/60% 0.1% H₃PO₄ to 90% ACN/10% 0.1% H₃PO₄ over 12 minutes, hold 3 minutes then back to 40% ACN over 1 minute. Retention times: amidoxime d—2.4 minutes, DMAD—6.7 minutes, intermediate adducts —8.4 and 8.6 minutes (8.4 minute peak cyclizes faster), product e —5.26 minutes, xylenes—several peaks around 10.4-10.7 minutes.

Step 5: N-Methylation

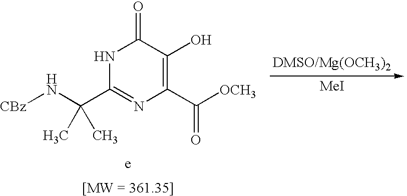

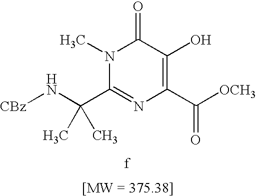

| Material | MW | Eq. | Mass | Volume |
|---|---|---|---|---|
| pyrimidine diol (e) | 361.35 | 1 | 2 kg | |
| Mg(OMe)₂, 8 wt. % in MeOH | | 2 | 11.95 kg | 13.4 L |
| MeI | | 4 | 3.14 kg | 1.38 L |
| DMSO | | | | 16 L |
| 2M HCl | | | | 20 L |
| MeOH | | | | 14 L |
| Na bisulfite 5 wt. % in water | | | | 2 L |
| water | | | | 60 L |

To a solution of the pyrimidine diol e (2 kg) in DMSO (16 L) was added a solution of Mg(OMe)₂ in MeOH (11.95 kg), after which excess MeOH was evaporated under vacuum (30 mm Hg) at 40° C. for 30 minutes. The mixture was then cooled down to 20° C., after which MeI (1.38 L) was added and the mixture stirred at 20-25° C. for 2 hours, and then at 60° C. for 5 hours under pressure in a closed flask. HPLC showed that the reaction was complete. The mixture was then cooled to 20° C., after which MeOH (14 L) was added, followed by the slow addition of 2 M HCl (20 L) [exotherm] over 60 minutes. Sodium bisulfite (5 wt. %, 2 L) was then added to quench excess I₂, with the solution turning white. Water (40 L) was then added over 40 minutes and the slurry stirred for 40 minutes in an ice bath, and then filtered. The filter cake was washed first with water (20 L) and then with MTBE:MeOH 9/1 (30 L) to remove O-methylated by-product. HPLC showed less than 0.5 A % O-methylated product after washing. The solid was dried overnight at room temperature under vacuum with an N₂ stream to give 1.49 kg of N-methyl pyrimidone (70% yield, corrected for purity of starting material and product).

Step 6: Amine coupling

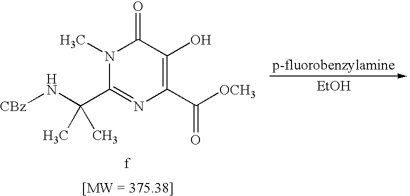

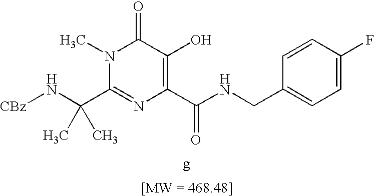

| Material | MW | Eq. | Mass | Volume |
|---|---|---|---|---|
| N-methylpyrimidinone (f) | 375.38 | 1 | 1.4 kg | |
| 4-fluorobenzylamine | 125.15 | 2.2 | 1.05 kg | |
| EtOH | | | | 14 L |
| water | | | | 14 L |
| acetic acid | | | | 0.55 L |

To a slurry of N-methylated pyrimidinone f (1.4 kg) in EtOH (14 L) at 4° C. was slowly added 4-fluorobenzylamine (1.05 kg) over 15 minutes, wherein an exotherm to 9° C. was observed during addition of the first 1 mole equivalent of the amine. The slurry became very thick and vigorous stirring was required. The reaction was warmed to 72° C. over 2 hours and maintained at this temperature for 1 hour and 45 minutes. The solution became extremely viscous at 45° C. where a small exotherm was observed to 50° C., after which the slurry slowly freed up and became homogeneous after 1 hour at 72° C. An HPLC sample assay (HPLC method was similar to that employed in Step 4 above) at the end of the reaction showed less than 0.5 A % N-methylated pyrimidinone. The reaction was then cooled to 60° C. and acetic acid (0.55L) was added over 30 minutes, followed by the addition of water (6.7 L) over 30 min and then the addition of seed (3.0 g) to initiate crystallization. After 30 min at 60° C., more water (7.3 L) was added over 30 minutes and the reaction mixture allowed to cool to ambient temperature overnight. After 13 hours, the temperature was at 20° C., at which point the reaction mixture was filtered and the slurry washed with 50% water/EtOH (2×4 L). The solids were dried on the filter pot under vacuum/N₂ flow to a constant weight to afford a white solid product (1.59 kg; 90% corrected yield; 99% LCWP and 99.7% LCAP as determined by HPLC method similar to that employed in Step 4 above.)

Step 7: Hydrogenation of Cbz-amide

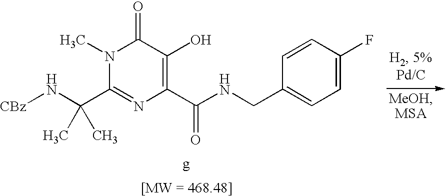

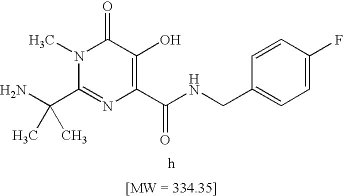

| Material | MW | mmoles | Mass | Volume | |
|---|---|---|---|---|---|
| CBz amide (g) | 468.48 | 21.33 | 10 g | | |
| MeOH | | | | 80 | mL |
| 5% Pd/C (50% wet) | | | 0.15 g | | |
| MSA | 96.1 | 22.4 | | 1.45 | mL |
| water | | | | 8 | mL |
| cake wash (4:1 MeOH:H₂0 | | | | 20 | mL |
| 1 N NaOH | | 22.4 | | 22.4 | mL |
| final cake wash (water) | | | | 30 | mL |

A stainless steel hydrogenation vessel was preconditioned with MeOH, Pd/C catalyst and MSA under the reaction conditions described below. Cbz-amide g (10 g) was then slurried in MeOH (80 mL) in the preconditioned vessel. MSA (1.45 mL) was added to the slurry in one portion at room temperature. 5% Pd/C (0.15 g, 50% wet) was also added to the hydrogenation vessel. Hydrogen was charged to the vessel in three successive vacuum/hydrogen purge cycles, after which the mixture was hydrogenated at 40 psig for 3-4 hour at 50° C. Following hydrogenation, water (8 mL) was added to the reaction mixture, the mixture was stirred, and the catalyst was filtered and washed with 4:1 MeOH:water (20 mL). The pH of combined filtrates was adjusted to pH 7 to 8.0 by slow addition of 1 N NaOH (22.4 mL), which precipitated a solid. The slurry was stirred at 0-5° C. for 4 hours and the solid filtered, washed with water (30 mL), collected and dried in vacuo at 50° C. The product amine (as hydrate) was obtained as a white crystalline solid (7.7 g) in 96% yield (corrected for KF), 89%LCWP, 99.8% LCAP, KF=11 wt. %

HPLC Method A (product assay): column: 25 cm×4.6 mm Zorbax RX-CS; mobile phase: A=0.1% H₃PO₄, B═CH₃CN, O minutes (80% A/20% B), 20 minutes (20% A/80% B), 25 minutes (20% A/80% B); flow: 1.0 mL/minute; wavelength: 210 nm; column temperature: 40° C.; retention times: desfluoroamine byproduct—5.5 min, amine product—5.85 minutes, toluene—16.5 minutes, Cbz-amide—16.82 minutes.

HPLC Method B (product purity): column: 25 cm×4.6 mm YMC-basic; mobile phase: A=25 mmol KH₂PO₄ adjusted to pH=6.1, B═CH₃CN, O minutes (90% A/10% B), 30 minutes (30% A/70% B), 35 minutes (30% A/70% B); flow: 1 mL/minute; wavelength: 210 nm; column temperature: 30° C.; retention times: des-fluoroamine—9.1 minutes, amine—10.1 minutes, toluene—24.2 minutes, Cbz amide—25.7 minutes.

Step 8: Oxadiazole Coupling
Part A: Preparation of Oxadiazole K Salt

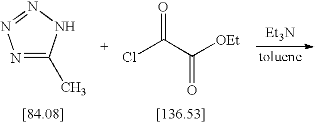

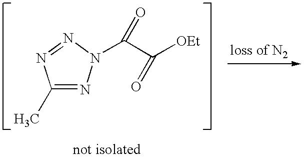

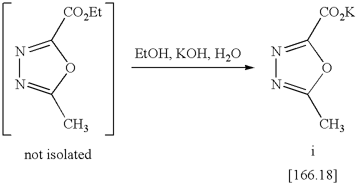

| Material | Eq. | Mole | Mass | Volume | Density |
|---|---|---|---|---|---|
| 5-methyltetrazole | 1.0 | 28.54 | 2.5 kg | | |
| (96 wt. %) | | | (2.4 kg) | | |
| ethyloxalyl chloride | 1.03 | 29.4 | 4.014 kg | 3.29 L | 1.22 |
| triethylamine | 1.05 | 29.97 | 3.033 kg | 4.21 L | 0.72 |
| toluene | | | | 74 L | |
| EtOH (punctilious) | | | | 61 L | |
| MTBE | | | | 15 L | |
| KOH aq. *20 wt. %) | | | | 8 L | |
| 10% brine | | | | 5 L | |

(Note:
An alternative procedure is included parenthetically. Except as noted therein, the alternative procedure is essentially the same as the main procedure.)

Ethyl oxalylchloride (4.01 kg) was slowly added to a mixture of 5-methyltetrazole (2.50 kg), triethylamine (3.03 kg) in toluene (32 L) at 0° C. at such a rate that the temperature stays below 5° C. The resulting slurry was stirred for 1 hour at 0-5° C. then the triethylamine/HCl salt was filtered off. (Note: In the alternative procedure, after stirring for 1 hour the slurry was heated to 60-65° C. over 1 hour with N₂ gas evolution and then aged at 60-65° C. for 1 hour and then cooled to 20-25° C. before recovering the triethylamine/HCl salt.) The solid was washed with 27 L of cold toluene (5° C.) (Note: In the alternative procedure noted above, the toluene was not cold.) The combined filtrates were kept at 0° C. and were slowly added to a hot solution of toluene (50° C., 15L) over 40-50 minutes (N₂ gas evolution), then the solution was aged at 60-65° C. for 1 hour. After cooling at 20° C., the toluene solution was washed with 5 L of 10% brine (Note: In the alternative procedure, the combined filtrates were only washed with brine.), then solvent switched to ethanol (reduced to 8 L, then 17 L of EtOH was added, then concentrated down to 8 L, then 33 liters of EtOH were added to adjust final volume of 41 L). The ethanol solution was cooled to 10° C. and KOH aq. (8.0 L) was added over 30 minutes, and the resulting thick slurry was then stirred for 40 minutes at room temperature while the oxadiazole K salt crystallized out. The solid was filtered off, washed with 11 L of EtOH and finally with 15 L of MTBE. The solid was dried overnight under vacuum at 20° C. with a nitrogen stream to yield 4.48 kg (90.8%) of the K-salt i.

Part B: Oxadiazole Coupling

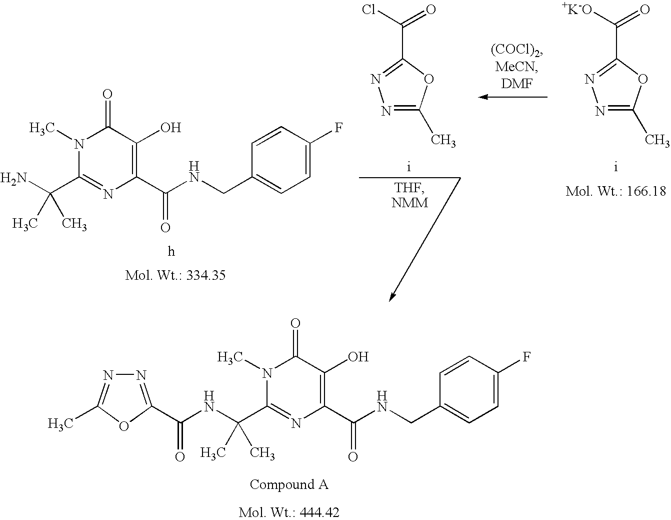

| Reagent | Mass | mL | | Moles | Eq. |
|---|---|---|---|---|---|
| oxadiazole K salt i | 33.8 g (96.1 wt %) | | | 0.20 | 2.2 |
| ACN | | 280 | mL | | |
| DMF | 0.33 | | | | |
| oxalyl chloride | 23.7 g | 16.3 | mL | 0.19 | 2.1 |
| free amine h | 30 g (99 wt %) | | 0.089 | 1 | |
| THF | | 821 | mL | | |
| NMM | 21.56 g | 23.4 | mL | 0.21 | 2.4 |
| NH₄OH (30% in H₂O) | 62.3 g | 69 | mL | 0.53 | 6 |
| HCl (2N) | | 500 | mL | | |
| IPA | | 920 | mL | | |
| water | | 400 | mL | | |
| MeOH | | 300 | mL | | |

(Note:
An alternative procedure is included parenthetically. Except as noted therein, the alternative procedure is essentially the same as the main procedure.)

A500 mL round bottom flask was charged with oxadiazole K salt i (33.8 g) followed by ACN (280 mL) and DMF (0.33 mL) with strong stirring. The resulting slurry was then cooled down to 0-5° C. and oxalyl chloride (23.7 g) was added over the course of 20 minutes in order to maintain the internal temperature at less than 5° C. The resulting acyl chloride-containing slurry was then aged for 1 hour.

To a 2 L round bottom flask the free amine h (30 g) was added followed by THF (821 mL). The resulting slurry was cooled down to 0-5° C., after which NMM (21.56 g) was added and the slurry so obtained was stirred for 10 minutes at the cold temperature. The previously prepared acyl chloride-containing slurry was added slowly to the free amine slurry over the course of 20 minutes such that the temperature did not exceed 5° C. The slurry was then aged for 1.5 hours at 0-5° C. At this time HPLC showed no more amine h (<0.5% LCAP, 100% conversion). The reaction mixture was then quenched with NH₄OH (30% in water) (69 mL) which was added over the course of 3 minutes (in the alternative procedure, aqueous KOH was employed in place of NH₄OH). The resulting yellow slurry was then stirred for an additional hour at temperatures less than 10° C. The yellow slurry was then acidified to pH 2-3 with HCl (2N) (500 mL). To the resulting red wine colored solution, IPA (920 mL) was added. The low boiling point organic solvents were then evaporated under reduced pressure (40 torr) at room temperature to a final solution volume of 1100 mL, at which volume crystalline Compound A began to precipitate. Water (400 mL) was then added to this new slurry over the course of 10 minutes, and the slurry aged overnight at room temperature (in the alternative procedure, the slurry was cooled to 0-10° C. and then aged for 2 hours). The aged slurry was filtered and the solid obtained was washed with water (170 mL), followed by a swish wash with cold MeOH (300 mL, previously cooled in an ice bath), and finally by a swish wash with water (700 mL). (In the alternative procedure, the solid obtained by filtering the aged slurry was washed twice with water; i.e., MeOH was not employed.) The solid so obtained was dried overnight under vacuum and nitrogen stream to give 35.5 g of Compound A (91% yield). (The alternative procedure provided Compound A in 95% yield.)

Step 9: Formation of a Crystalline Potassium Salt of Compound A

Acetonitrile (50 mL) and anhydrous Compound A (5.8 g, 97.4 wt. %) were charged at room temperature to a jacketed 125 mL round bottom flask equipped with a mechanical stirrer and equipped with a nitrogen inlet (i.e., the crystallization was conducted under nitrogen). The resulting slurry was agitated at 45° C. until the solids were completely in solution. Form 1 crystalline Compound A K salt was then charged to the solution as seed (0.184 g, 3 wt % to theoretical K salt). Aqueous KOH 30% w/v solution (0.98 eq., 2.33 mL, 0.0125 moles) was then added with the following charge profile while maintaining batch at 45° C.:

0.466 mL over 5 hours, 0.0932 mL/hr (20 mol %)

1.864 mL over 7 hours, 0.2663 mL/hr (80 mol %)

The resulting slurry was cooled to 20° C. and aged at 20° C. until the concentration of Compound A in the mother liquor was measured to be less than 4 g/L. The batch was filtered, the cake washed with ACN (3×12 mL), and then dried under vacuum at 45° C., with a small nitrogen sweep, until the amount of ACN and water present as determined by thermogravimetric analysis was less than 1 wt. %. The K salt of Compound A was obtained in >99 A % by HPLC analysis.

### EXAMPLE 2

Form 1 Crystalline Potassium Salt of Compound A

Part A: Preparation

Ethanol ( 147 mL), water ( 147 mL), and Compound A ( 97.9 g assay by HPLC) were charged to a 1 L round bottom flask equipped with mechanical stirrer, addition funnel, nitrogen inlet (i.e., run conducted under nitrogen), and a thermocouple. Aqueous KOH (45% w/w, 0.98 eq., 18.5 mL, 216 mmoles) was added to the suspension over 10 minutes at 21° C. The resulting suspension was agitated for 0,5 hour resulting in the dissolution of a majority of the solids, after which the batch was filtered through a 1 μm filter directly into a 5 L round bottom flask equipped with mechanical stirrer, addition funnel, nitrogen inlet, and thermocouple. The 1 L flask was rinsed with 1:1 (v/v) water/EtOH (48 mL) and the rinse was filtered into the 5 L crystallization vessel. The filtered solution was seeded with crystalline Form 1 Compound A K salt (200 mg) at room temperature and then aged for 1 hour to build a good seed bed, after which the suspension was diluted with EtOH (1.57 L) at 20° C. over 1.5 hour The batch was then cooled to about 4° C. and aged until the concentration of Compound A in the mother liquor was measured to be 4.7 g/L. The batch was filtered, the crystallization vessel rinsed with 50 mL EtOH into the filter, the cake washed with EtOH (4×100 mL), and then dried under vacuum and a nitrogen tent until the amount of EtOH present by NMR was about 0.4 mol % relative to the potassium salt. The potassium salt of Compound A was obtained in 88% yield ( 91.5 g assay by HPLC, 99 area % by HPLC analysis).

Part B: Characterization

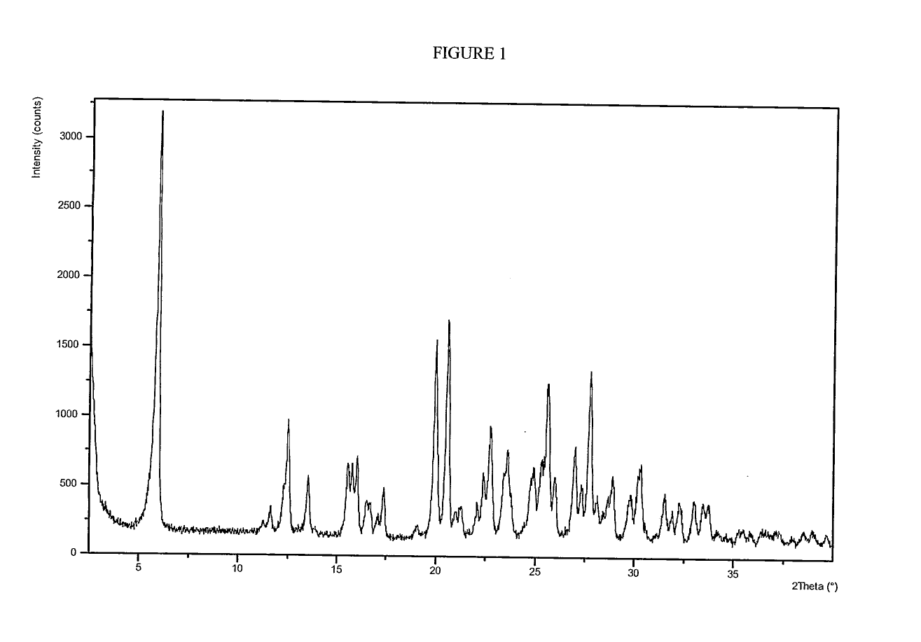

An XRPD pattern of a K salt prepared in the manner described in Part A was Generated on a Philips Analytical X'Pert Pro X-ray powder diffractometer using a continuous scan from 2.5 to 40 degrees 3 Θ over about 12 minutes (i.e., 0.02° step size with 40 seconds/step), 2 RPS stage rotation, and a gonio scan axis. Copper K-Alpha 1 (K_{α1}) and K-Alpha 2 (K_{α2}) radiation was used as the source. The experiment was run under ambient conditions. Characteristic 2Θ values in the XRPD pattern (shown in FIG. 1) and the corresponding d-spacings include the following:

| Peak No. | d-spacing (Å) | 2 Theta |
|---|---|---|
| 1 | 14.9 | 5.9 |
| 2 | 7.1 | 12.5 |
| 3 | 4.4 | 20.0 |
| 4 | 4.3 | 20.6 |
| 5 | 3.5 | 25.6 |

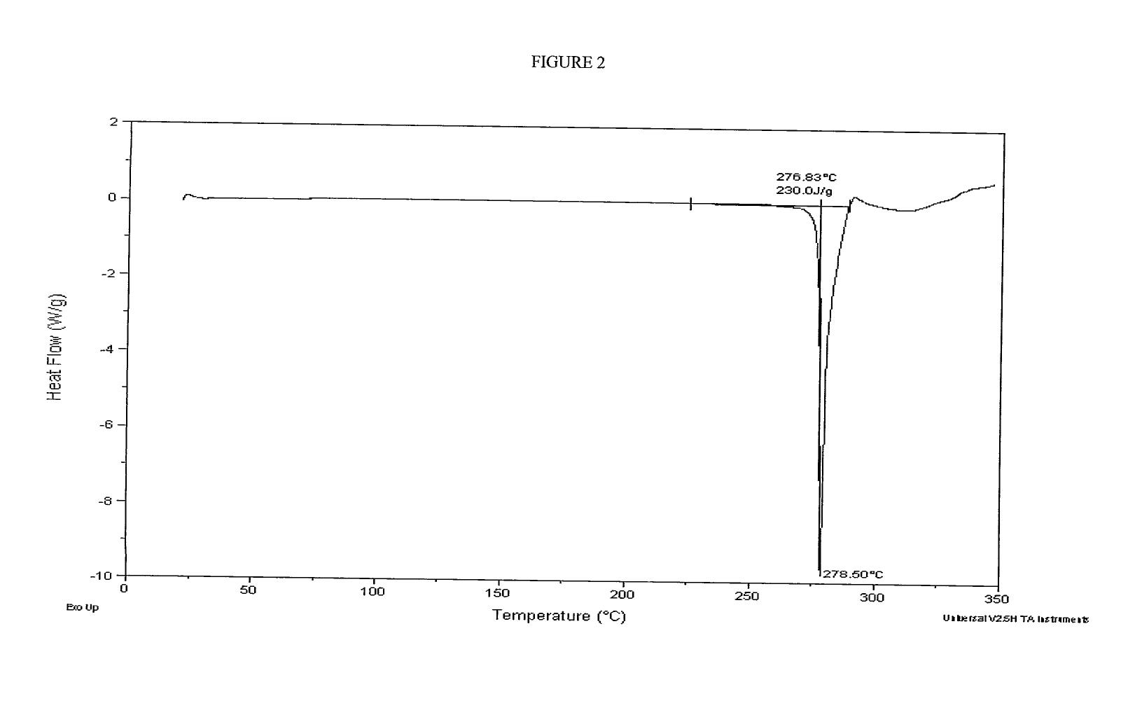

A K salt prepared in the manner described in Part A was also analyzed by a TA Instruments DSC 2910 differential scanning calorimeter at a heating rate of 10° C./min from room temperature to 350° C. in a crimpled pinhole aluminum pan in a nitrogen atmosphere. The DSC curve (shown in FIG. 2) exhibited a single, sharp endotheim with a peak temperature of about 279° C. and an associated heat of fusion of about 230.0 J/gm. The endotherm is believed to be due to melting.

A thermiogravimetric analysis was performed with a Perkin-Elmer Model TGA7 under nitrogen at a heating rate of 10° C./min from room temperature to about 350° C. The TG curve showed a 0.3% weight loss during heating to 250° C.

Hygroscopicity data was obtained on a VTI Symmetrical Vapor Sorption Analyzer Model SGA-1. Data was collected at room temperature from 5-95% relative humidity and back, 5% relative humidity change per step. Equilibrium conditions were 0.01 weight percent change in 5 minutes with a maximum equilibration time of 180 minutes. The data indicated that the material had a 1.8% weight increase when equilibrated at 95% RH at 25° C. When equilibrated back down to 5% RH, the material returned back to approximately its dry weight. An XRPD analysis of the material after the hygroscopicity experiment showed that the material had not changed phases.

K salt prepared as described in Part A was also assayed by HCl titration using a Brinkmann Metrohm 716 DMS Titrino. The assay results indicated the salt was a monopotassium salt.

### EXAMPLE 2-A

Form 1 Crystalline Potassium Salt of Compound A

Compound A (400 g) was dissolved in 4 liters of 60:40 ethanol:acetonitrile at 45° C. to provide a solution of Compound A with a concentration of 95 g/L. Ethanol (1201 g) was added to 300 g of a 24 wt. % solution of potassium ethoxide in ethanol to obtain a 4.8 wt % solution of KOEt in ethanol. A seed bed was prepared by adding Form 1 crystalline potassium salt of Compound A (78 g) to 1.08 liters of 70:30 ethanol:aceontitrile. The seed bed was wet milled using an Ultra Turrax IKA T-50 mixer for 45 minutes at 10,000 rpm, reaching 50,000 particle counts (1-500 um) and a mean particle size of 10 um as determined with a Lasentec FBRM Model S400 particle size analyzer.

The seed slurry (1.16 liters) was charged to a crystallizer with a jacket temperature set to 35° C. The solution of Compound A at 45° C. was then charged to the seed slurry in the crystallizer. While agitating the Compound A solution-seed slurry at 250 rpm, the KOEt solution was charged to the crystallizer above the surface of the solution-seed slurry at a constant rate of 4.7 mL/minutes over 6 hours and 40 minutes. The crystallizer jacket temperature was set to 35° C. for the first 6 hours and then changed to 20° C. while the remaining ˜9% of ethoxide was charged over the last 40 minutes. The batch was aged at 20° C. for 30 minutes and filtered, and the resulting filter cake was washed with 2.8 L of ethanol. The washed cake was then blown with nitrogen for 1 hour and transferred to a vacuum oven and dried overnight at 45° C. to afford the title salt.

### EXAMPLE 3

Preparation of Compressed Tablets Containing Compound A Potassium Salt

Part A

| | Amount per Tablet | Amt per batch |
|---|---|---|
| Ingredient | (mg) | (wt. percent) |
| Compound A K salt¹ | 111.2 | 27.8 |
| (on free phenol basis) | (100) | (25.0) |
| microcrystalline cellulose | 189.6 | 47.4 |
| (AVICEL PH-102) | | |
| lactose monohydrate | 63.2 | 15.8 |
| croscarmellose sodium | 12.0 | 3.0 |
| HPMC 2910 (6 centipoise) | 20.0 | 5.0 |
| magnesium stearate (intragranular) | 2.0 | 0.5 |
| magnesium stearate (extragranular) | 2.0 | 0.5 |

¹Form 1 crystalline monopotassium salt of Compound A; conversion factor (including purity) = 1.112.

Compressed tablets containing 100 mg of Compound A on a free phenol basis were prepared by blending all of the ingredients listed above, except for the extragranular magnesium stearate, in a blender (Turbula® Type T2F shaker-mixter, Basel, Switzerland) for 10 minutes. Portions of the blended material weighing approximately 1 gram were compressed into compacts (or slugs) in a benchtop press (Auto Carver Model Auto “C”, Catalog No. 3888, Carver, Inc., Wabash, Ind.) using 1×0.5 inch rectangular tooling to 12 MPa (4 KN). The slugs were then sized into granules by passing them through a sieve with 1 mm openings. The granules were blended with the extragranular magnesium stearate in the Turbula blender for 5 minutes, and the lubricated granules were compressed into tablets using the Auto Carver press with 13/32-inch standard concave round tooling.

Part B

| | Amount per Tablet | Amt per batch |
|---|---|---|
| Ingredient | (mg) | (wt. percent) |
| Compound A K salt¹ | 110 | 27.5 |
| (on free phenol basis) | (100) | (25.0) |
| microcrystalline cellulose | 175.2 | 43.8 |
| (AVICEL PH-102) | | |
| microcrystalline cellulose | 9.2 | 2.3 |
| (AVICEL PH-105) | | |
| lactose monohydrate | 61.6 | 15.4 |
| croscarmellose sodium | 12.0 | 3.0 |
| HPMC 2910 (6 centipoise) | 20.0 | 5.0 |
| magnesium stearate (intragranular) | 4.0 | 1.0 |
| magnesium stearate (extragranular) | 8.0 | 2.0 |

¹Form 1 crystalline monopotassium salt of Compound A; conversion factor (including purity) = 1.112.

Compressed tablets having the composition set forth in the above table were prepared using a procedure similar to that set forth in Part A.

### EXAMPLE 4

Preparation of Compressed Tablets Containing Compound A Potassium Salt

| | Amount per Tablet | Amt per batch |
|---|---|---|
| Ingredient | (mg) | (wt. percent) |
| Compound A K salt¹ | 434.4 | 50.0 |
| (on free phenol basis) | (400) | (46.0) |
| microcrystalline cellulose | 112.9 | 13.0 |
| (Avicel PH102) | | |
| lactose hydrous spray dried | 26.06 | 3.0 |
| anhydrous dibasic calcium | 73.85 | 8.50 |
| phosphate | | |
| HPMC K4M | 26.06 | 3.0 |
| poloxamer 407 (micronized grade)² | 173.8 | 20.0 |
| sodium stearyl fumarate | 8.69 | 1.0 |
| magnesium stearate | 13.03 | 1.50 |

¹Form 1 crystalline monopotassium salt of Compound A; conversion factor = 1.086.
²Obtained from BASF. Median particle size = 50 μm.

Compressed tablets containing 400 mg of Compound A on a free phenol basis were prepared by a roller compaction and tablet compression process train. Poloxamer 407, magnesium stearate, and sodium stearyl fumarate were pre-screened through No. 30 and No. 60 mesh size screens in succession, and then blended with all of the other ingredients, except for the extragranular magnesium stearate, in a Patterson-Kelly (PK) V-blender for 5 minutes. The blended material was then sieved through a No. 35 screen mesh to break up agglomerates, and the sieved material was then blended further in the same PK blender for about 15-20 minutes. The blend was then roller compacted using a Freund Type TF mini roller compactor at a roll pressure of 40 Kgf/cm², roll speed of 3 rpm and screw speed of 10 rpm. The resulting ribbon was milled in a small Quadro Comil fitted with a round impeller, screen size 39R (i.e., round hole size 0.039 inches; approximately mesh size No. 20) and operated at 1700 rpm. The resulting granules were then blended with 0.5% extragranular magnesium stearate in the PK blender for 5 minutes to produce the final blend. The lubricated granules were then compressed into tablets using a rotary tablet press with plain oval shaped tooling at a compression force necessary to achieve a tablet hardness of 16 to 20 kiloponds (i.e., 156.9 to 196.1 Newtons) as measured by using a Key model HT-300 hardness tester.

### EXAMPLE 5

In vitro Studies

The IC₅₀ value of atazanavir for inhibition of glucuronidation of Compound A by human liver microsomes was determined using a pool of human liver microsomes (obtained from Xenotech LLC, Lenexa, Kans.). Compound A (as the potassium salt) was added to human liver microsomes (1.0 mg/mL) in a buffer consisting of 0.1 M potassium phosphate buffer (pH=7.4), 5 mM magnesium chloride, 10 μg/mL alamethicin, and 10 mM D-saccharic acid 1,4-lactone. The mixture of Compound A and the buffered microsomes (0.5 mL) was incubated at Compound A's Kₘ (200 μM). UDPGA was added to the incubated sample to a concentration of 4 mM to initiate the glucuronidation reaction, which was stopped after 25 minutes (37° C.) with 2 volumes (i.e., 1 mL) of acetonitrile containing 1.5 μM of Compound B as an internal standard for the subsequent LC/MS analysis. Each of the samples was then centrifuged and the resulting supernatant was diluted 1:1 with 0.1% formic acid in water and a 10 μL aliquot was injected onto the LC/MS to determine the amount of glucuronide formation. Analogous samples of Compound A containing concentrations of atazanavir ranging from 0.1 to 50 μM were prepared, incubated, and tested in the same manner.

The IC₅₀ value of atazanavir for inhibition of glucuronidation of Compound A in the presence of rat liver microsomes was determined using a procedure similar to that just described above for human liver microsomes.

The IC₅₀ value of atazanavir for Compound A inhibition in human liver microsomes was found to be 0.5 μM. Atazanavir was also found to inhibit the glucuronidation of Compound A by rat liver microsomes (41% at a concentration of 50 μM).

### EXAMPLE 6

In Vivo Rat Studies

Male Sprague-Dawley rats (4 rats/group) weighing approximately 300 grams each were given orally either 0.5% methylcellulose in water (control group) or atazanavir in 0.5% methylcellulose once a day for three days. The atazanavir daily doses were 50 mg/kg and were administered in 0.5% methylcellulose at 5 mL/kg. On day four the control rats were dosed with 10 mg/kg of Compound A in the form of a potassium salt in 0.5% methylcellulose while the treatment group received atazanavir followed by an oral dose of 10 mg/kg of Compound A (as potassium salt) in 0.5% methylcellulose. Blood samples were taken from all rats in treatment and control groups at 0, 0.25, 0.5, 1, 2, 4, 6, 8, and 24 hours post dose on day 4. Plasma levels of Compound A were determined by LC-MS/MS as follows:

UDP-glucuronosyltransferase activity was determined by measuring the formation of the glucuronide of Compound A. HPLC analysis was carried out on an Agilent HP 1100 gradient system using the following parameters: column=Phenomenex Luna C18-2 (2 mm×150 mm, 5 μm); mobile phase=0.1% formic acid in water (solvent A) and 0.1% formic acid in acetonitrile (solvent B); flow rate=0.2 mL/min; procedure=the initial solvent composition of 10% B was increased to 80% B over 10 minutes, followed by holding solvent B constant at 80% for 3 minutes, and then returning to initial conditions for 6 minutes. The HPLC system was interfaced with a Finnigan TSQ Quantum tandem mass spectrometer. Mass spectral analyses were carried out using electrospray ionization (ESI) in the positive ion mode. The temperature for the ion transfer tube was 320° C. and the ESI ionizing voltage was maintained at 4.4 kV for all analyses. Tandem mass spectrometry (MS/MS) was based on collision-induced dissociation (CID) of ions entering the rf-only octapole region where argon was used as the collision gas at a pressure of 0.8 mtorr. A collision offset at −22 eV was used for MS/MS analyses. The CID transitions used were m/z=621.1→445.1 (Compound A glucuronide) and 431.2→306.1 (Compound B).

The Cₘₐₓ and AUC values for the rats receiving the oral dose of atazanavir (50 mg/kg) and Compound A (10 mg/kg) on day 4 were 7.2±6.1 μM and 9.9±3.7 μM·hr respectively. The corresponding values for the Compound A control group were 2.3±0.9 μM and 2.9±0.6 μM·hr respectively. Thus, atazanavir increased the plasma levels of Compound A by about 3-fold.

### EXAMPLE 7

In Vivo Human Studies

The protocol was a 2-period, fixed sequence study in healthy human male volunteers to examine the influence of multiple doses of atazanavir on a single dose of Compound A. In Period 1, 12 subjects received a single oral dose of 100 mg of Compound A (i.e., a tablet as described in Part B of Example 3) (N=10) or placebo (N=2). In Period 2, the same 12 subjects were administered 400 mg atazanavir once daily in an open-label fashion (capsules) for 9 days. On Day 7, the subjects were administered atazanavir in combination with a single oral dose of 100 mg of Compound A (tablet) or placebo (the same subjects received placebo in both study periods). All doses were administered following a moderate-fat meal. Plasma PK samples were collected for 72 hours following the dose of Compound A in both periods.

Sample preparation and analysis: The plasma samples were extracted using 96-well liquid-liquid extraction. Plasma extracts were injected onto an Ace C₁₈ (50×3.0 mm, 3 μm, titanium rits) HPLC column and analyzed under isocratic conditions with a mobile phase consisting of 42.5/57.5 (v/v %) 0.1 mM EDTA in 0.1% formic acid/methanol, at a flow rate of 0.5 mL/minute. The sample extracts were ionized using an APCI interface and were monitored by MRM in the positive ionization mode. The dynamic range of the LC/MS/MS assay was 2-1000 ng/mL based on a 200 μL aliquot of human plasma.

PK Calculations: Area under the curve for a plot of plasma concentration v. time to last detectable concentration (AUC₀₋ₗₐₛₜ), was calculated using a non-compartmental model and the Linear Up/Log Down calculation method in WinNonlin Version 4.1. Data points after Cₘₐₓ were fitted to a biexponential equation (A*exp(−αt)+B*exp(−βt)) using WinNonlin v4.1, and AUC values were extrapolated to infinity according to the following equation: AUC₀₋₂₈=AUC₀₋ₗₐₛₜ+Cₗₐₛₜ/β, where Cₗₐₛₜ is the last detectable concentration and β comes from the above-noted biexponential equation. Observed maximum plasma concentration (Cₘₐₓ), time of Cₘₐₓ (Tₘₐₓ), and plasma concentration at 12 hr post dosing (C₁₂ₕᵣ) were determined by inspection.

Results: Higher plasma levels of Compound A were observed in the presence versus absence of atazanavir. The geometric mean ratio (GMR) 12 hr concentration in the presence versus absence of atazanavir was 1.96. Higher AUC and Cₘₐₓ values were also observed for Compound A in the presence versus absence of atazanavir [for AUC₀₋ₗₐₛₜ, GMR=1.73; for Cₘₐₓ, GMR=1.53]. There was also a trend toward a slightly longer alpha phase half-life for Compound A in the presence versus absence of atazanavir (1.4 hours in the presence of atazanavir versus 1.1 hours for Compound A alone).

While the foregoing specification teaches the principles of the present invention, with examples provided for the purpose of illustration, the practice of the invention encompasses all of the usual variations, adaptations and/or modifications that come within the scope of the following claims.

1. A method for improving the pharmacokinetics of an orally administered drug that is directly metabolized by UGT1A1 which comprises orally administering to a mammal in need of treatment with the drug an effective amount of a combination of the drug or a pharmaceutically acceptable salt thereof and atazanavir or a pharmaceutically acceptable salt thereof.

2. The method according to claim 1, wherein the drug that is directly metabolized by UGT1A1 is a compound of Formula I, or a pharmaceutically acceptable salt thereof: 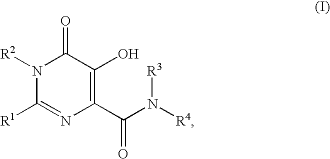
wherein R¹ is C₁₋₆ alkyl substituted with:
(1) N(R^{A})—C(═O)—N(R^{C})R^{D},
(2) N(R^{A})—C(═O)—C₁₋₆ alkylene-N(R^{C})R^{D},
(3) N(R^{A})SO₂R^{B},
(4) N(R^{A})SO₂N(R^{C})R^{D},
(5) N(R^{A})—C(═O)—C₁₋₆ alkylene-SO₂R^{B},
(6) N(R^{A})—C(═O)—C₁₋₆ alkylene-SO₂N(R^{C})R^{D},
(7) N(R^{A})C(═O)C(═O)N(R^{C})R^{D},
(8) N(R^{A})—C(═O)-HetA,
(9) N(R^{A})C(═O)C(═O)-HetA, or
(10) HetB;
R² is —C₁₋₆ alkyl;
or alternatively R¹ and R² are linked together such that the compound of Formula I is a compound of Formula II: 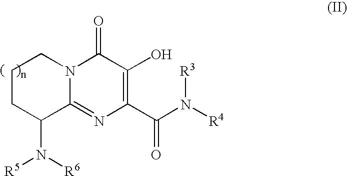
R³ is —H or —C₁₋₆ alkyl;
R⁴ is C₁₋₆ alkyl substituted with an aryl, which is optionally substituted with from 1 to 4 substituents each of which is independently halogen, —OH, —C₁₋₄ alkyl, —C₁₋₄ alkyl-OR^{A}, —C₁₋₄ haloalkyl, —O—C₁₋₄ alkyl, —O—C₁₋₄ haloalkyl, —CN, —NO₂, —N(R^{A})R^{B}, —C₁₋₄ alkyl-N(R^{A})R^{B}, —C(═O)N(R^{A})R^{B}, —C(═O)R^{A}, —CO₂R^{A}, —C₁₋₄ alkyl-CO₂R^{A}, —OCO₂R^{A}, —SR^{A}, —S(═O)R^{A}, —SO₂R^{A}, —N(R^{A})SO₂R^{B}, —SO₂N(R^{A})R^{B}, —N(R^{A})C(═O)R^{B}, —N(R^{A})CO₂R^{B}, —C₁₋₄ alkyl-N(R^{A})CO₂R^{B}, methylenedioxy attached to two adjacent ring carbon atoms, phenyl, or —C₁₋₄ alkyl-phenyl;
R⁵ is:
(1) N(R^{A})—C(═O)—N(R^{C})R^{D},
(2) N(R^{A})—C(═O)—C₁₋₆ alkylene-N(R^{C})R^{D},
(3) N(R^{A})SO₂R^{B},
(4) N(R^{A})SO₂N(R^{C})R^{D},
(5) N(R^{A})—C(═O)—C₁₋₆ alkylene-SO₂R^{B},
(6) N(R^{A})—C(═O)—C₁₋₆ alkylene-SO₂N(R^{C})R^{D},
(7) N(R^{A})C(═O)C(═O)N(R^{C})R^{D},
(8) N(R^{A})—C(═O)-HetA,
(9) N(R^{A})C(═O)C(═O)-HetA, or
R⁶ is —H or —C₁₋₆ alkyl;
n is an integer equal to 1 or 2;
each R^{A} is independently —H or —C₁₋₆ alkyl;
each R^{B} is independently —H or —C₁₋₆ alkyl;
R^{C} and R^{D} are each independently —H or —C₁₋₆ alkyl, or together with the nitrogen to which they are attached form a saturated 5- or 6-membered heterocyclic ring optionally containing a heteroatom in addition to the nitrogen attached to R^{C} and R^{D} selected from N, O, and S, where the S is optionally oxidized to S(O) or S(O)₂, and wherein the saturated heterocyclic ring is optionally substituted with 1 or 2 C₁₋₆ alkyl groups;
HetA is a 5- or 6-membered heteroaromatic ring containing from 1 to 4 heteroatoms independently selected from N, O and S, wherein the heteroaromatic ring is optionally substituted with 1 or 2 substituents each of which is independently —C₁₋₄ alkyl, —C₁₋₄ haloalkyl, —O—C₁₋₄ alkyl, —O—C₁₋₄ haloalkyl, or —CO₂R^{A}; and
HetB is a 5- to 7-membered saturated heterocyclic ring containing from 1 to 4 heteroatoms independently selected from N, O and S, wherein each S is optionally oxidized to S(O) or S(O)₂, and the heterocyclic ring is optionally substituted with from 1 to 3 substituents each of which is independently halogen, —C₁₋₄ alkyl, —C₁₋₄ fluoroalkyl, —C(O)—C₁₋₄ alkyl, or —C₁₋₄ alkyl substituted with OH.

3. The method according to claim 2, wherein the drug is Compound A, or a pharmaceutically acceptable salt thereof, wherein Compound A is:

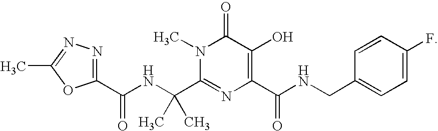

4. The method according to claim 3, wherein atazanavir is administered in the combination in an amount sufficient to improve the pharmacokinetics of Compound A by at least about 10% with respect to the pharmacokinetics of Compound A administered in the absence of atazanavir.

5. The method according to claim 3, wherein the amount of Compound A administered per day in the combination is in a range of from about 5 mg/kg to about 10 mg/kg of body weight and the amount of atazanavir administered per day in the combination is in a range of from about 2 mg/kg to about 10 mg/kg of body weight.

6. The method according to claim 3, wherein atazanavir is administered in the combination in an amount that, if administered alone, is less than that which is effective for treating HIV infection or AIDS.

7. The method according to claim 3, wherein the amount of Compound A administered per day in the combination is in a range of from about 5 mg/kg to about 10 mg/kg of body weight and the amount of atazanavir administered per day in the combination is in a range of from about 2 mg/kg to about 5 mg/kg of body weight.

8. The method according to claim 3, wherein the amount of Compound A administered per day in the combination is in a range of from about 5 mg/kg to about 10 mg/kg and the amount of atazanavir administered per day in the combination is less than 400 mg.

9. A pharmaceutical combination for oral administration to a mammal comprising a drug that is useful for the treatment or prophylaxis of a disease or condition and that is directly metabolized by UGT1A1, or a pharmaceutically acceptable salt thereof, and atazanavir or a pharmaceutically acceptable salt thereof, wherein the drug and atazanavir are each employed in an amount that provides therapeutic or prophylactic efficacy of the drug.

10. The combination according to claim 9, wherein the HIV integrase inhibitor that is directly metabolized by UGT1A1 is a compound of Formula I as set forth in claim 2, or a pharmaceutically acceptable salt thereof.

11. The combination according to claim 10, wherein the HIV integrase inhibitor that is directly metabolized by UGT1A1 is Compound A, or a pharmaceutically acceptable salt thereof, wherein Compound A is:

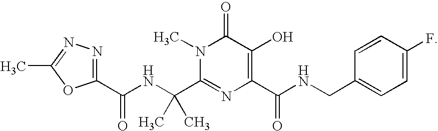

12. The combination according to claim 11, wherein atazanavir is administered in the combination in an amount sufficient to improve the pharmacokinetics of Compound A by at least about 10% with respect to the pharmacokinetics of Compound A administered in the absence of atazanavir.

13. The combination according to claim 11, wherein the amount of Compound A administered per day in the combination is in a range of from about 5 mg/kg to about 10 mg/kg of body weight and the amount of atazanavir administered per day in the combination is in a range of from about 2 mg/kg to about 10 mg/kg of body weight.

14. The combination according to claim 11, wherein atazanavir is administered in the combination in an amount that, if administered alone, is less than that which is effective for treating HIV infection or AIDS.

15. The combination according to claim 9, wherein the combination is a single pharmaceutical composition which further comprises a pharmaceutically acceptable carrier.

16. The method according to claim 3, wherein Compound A is employed in the form of a potassium salt.

17. The method according to claim 16, wherein atazanavir is administered in the combination in an amount sufficient to improve the pharmacokinetics of Compound A by at least about 10% with respect to the pharmacokinetics of Compound A administered in the absence of atazanavir.

18. The method according to claim 16, wherein the mammal is an adult human, the amount of Compound A administered per day in the combination is in a range of from about 200 mg to about 1200 mg and the amount of atazanavir administered per day in the combination is less than 400 mg.

19. The method according to claim 18, wherein the atazanavir is administered in an amount in a range of from about 100 mg to about 350 mg per day.

20. The combination according to claim 11, wherein Compound A is employed in the form of a potassium salt.
